(19) United States
(12) Patent Application Publication
(10) Pub. No.: US 2020/0240990 A1
(43) Pub. Date: Jul. 30, 2020

(54) REG3A AND REG FAMILY MEMBER BIOMARKERS AND METHODS FOR DIAGNOSIS AND TREATMENT OF CANCER

(71) Applicant: University of Kentucky Research Foundation, Lexington, KY (US)

(72) Inventors: Sabine Brouxhon, Lexington, KY (US); Stephanos Kyrkanides, Lexington, KY (US); Melvyn Yeoh, Lexington, KY (US); Ronald Bruntz, Lexington, KY (US); Matthew Hoover, Lexington, KY (US)

(21) Appl. No.: 16/751,791

(22) Filed: Jan. 24, 2020

Related U.S. Application Data
(60) Provisional application No. 62/796,177, filed on Jan. 24, 2019.

(51) Int. Cl. G01N 33/574; A61K 39/395; A61K 9/00

(52) U.S. Cl. CPC G01N 33/57438; G01N 33/57407; A61K 39/3955; G01N 2333/4724; A61K 9/0021; A61K 9/0053; A61K 9/0043; A61K 9/0019

(57) ABSTRACT

Methods for diagnosing a cancer in a subject include providing a biological sample from the subject and determining an amount of Reg3A in the sample. The subject is then diagnosed as having cancer or a risk thereof if there is a measurable difference in the amount of the Reg3A in the biological sample as compared to a control level of the Reg3A. The amount of Reg3A can be determined by itself or in combination with an amount of soluble E-cadherin (sEcad) and/or additional Reg family members. The subject can further be administered an effective amount of an agent capable of affecting an expression level or activity of Reg3A as part of a therapeutic method for treating a cancer. The Reg3A affecting agent can be an anti-Reg3A antibody that is administered alone or with an effective amount of a chemotherapeutic agent.

### RELATED APPLICATIONS

This application claims priority from U.S. Provisional Application Ser. No. 62/796,177, filed Jan. 24, 2019, the entire disclosure of which is incorporated herein by this reference.

### TECHNICAL FIELD

The presently-disclosed subject matter generally relates to compositions and methods for the diagnosis and treatment of cancer that make use of and/or target Reg3A and/or Reg-related family members. In particular, the compositions and methods of the presently-disclosed subject matter relate to biomarkers for early cancer detection, recurrence and prognosis, diagnostic kits, methods of detection, and/or therapeutic treatments with agents including binding agents, such as antibodies, as well as antigenic fragments useful for treating cancer, either alone or in combination with chemotherapy and/or other cancer therapeutics, such as agents that target the HGF/c-Met-FAK-Src pathway.

### BACKGROUND

Cancer is one of the leading causes of death worldwide. In the United States, an estimated 1,735,350 new cases of cancer were diagnosed, with 609,640 deaths in 2018. An increased understanding of the biology of cancer cells, combined with precision medicine and genomic and proteomic approaches have revolutionized the way diseases including cancer are treated, thereby leading to a new generation of FDA-approved therapies. Cancer biomarkers are also rapidly transforming patient outcomes, and are the foundation upon which successful precision medicine cancer treatments can be built. Thus, combining drug therapies with a proven biomarker have higher success rates than those without, and further allows for more efficient trials to be conducted in rare cancer types, thereby accelerating drug development. However, despite these advancements, cancer drug and biomarker development has been slow and extremely inefficient in identifying novel biomarker technology platforms for cancer diagnosis and therapies.

Regenerating islet-derived 3 alpha (Reg3A), also known as hepatocarcinoma-intestine-pancreas (HIP) or pancreatitis-associated-protein (PAP), is a member of a multifunctional family of secreted calcium-dependent proteins containing a C-type lectin-like domain linked to a short N-terminal peptide. As far as Reg family nomenclature, a great deal of confusion has been generated from the literature. However, according to recent genome sequencing, there are a total of five human Reg family member subclasses, including RegIA (Reg1A), RegIB (Reg1B), RegIIIA (Reg3A, HIP/PAP), RegIII (Reg3G) and RegIV (Reg4), with the Reg gene encoding a 166-amino acid peptide with a signal peptide of 23 amino acids. These Reg genes are believed to have evolved from a common ancestral species, exhibit a large degree of homology, and, with the exception of Reg4, are located at adjacent sites of chromosome 2p12.

Despite the large degree of homology in these Reg family members, distinct structural and functional differences have been reported. For example, Reg3A has three splice variants which all encode the same protein, whereas Reg3G also encodes three splice variants but encodes two distinct proteins. Moreover, Reg3A and Reg3G exhibit differential gene expression levels in human tissues, with Reg3A being the predominant family member in liver, stomach, small intestine, and brain, whilst Reg3G expression predominates in kidney, testis, and placenta. Along these lines, the ratio of Reg3A to Reg3G mRNA expression is similar in normal pancreas.

As far as Reg3A as a tumor-specific target, Reg3A has been shown to exert conflicting functional roles in select gastrointestinal cancers, including gastric (GC), pancreatic (PDAC), colorectal (CRC), and hepatocellular carcinomas (HCCs). Specifically, in GCs, Reg3A has been reported to either suppress or promote cancer cell proliferation, migration, and invasion. Moreover, treatment of PDAC cells with recombinant Reg3A promoted migration, whereas siRNA-mediated suppression of Reg3A decrease migration in HCC cells. Finally, Reg3A was shown to promote tumor vascularization as pancreatic tumors from Reg3A knock-out mice were significantly less vascularized compared to wild-type mice. Thus, it is not clear whether Reg3A may be a tumor-specific target in these GI tumor types, and its role in Head & Neck Cancers (HNSCCs) has yet to be reported in the literature, much less have there been reports in the literature as to whether therapeutic agents that target Reg3A and/or related Reg family members would be useful in treating cancer, or other pathological conditions.

Insofar as Reg3A as a tumor-specific biomarker, the literature is also conflicting, particularly with respect to GI cancers. Specifically, in GCs, Reg3A levels were upregulated in the peripheral blood of patients, whilst expression levels of Reg3A were down-regulated in primary cancer specimens. Moreover, quantitative mass spectrometry analysis of human plasma samples revealed that a biomarker combination, that included Reg3A and other moieties, could be utilized to detect pancreatic cancer patients versus normals. Similarly, using protein biochip technology, others have identified Reg3A in the pancreatic secretions of patients with PDAC, as well as other pancreatic diseases. Reg3A levels were further shown to be elevated in PDAC secretions as well as in chronic pancreatitis, and reported to correlate with tumor load. Lastly, as far as gene expression alterations, Reg3A has been found to be upregulated in infiltrating pancreatic cancers compared to adjacent non-neoplastic parenchyma, but the primers used for qPCR might also amplify Reg3G, and be non-selective for these different Reg3 family members. In HCCs, both mRNA and protein expression of Reg3A were upregulated in tumors compared to adjacent normal tissues, and correlated with a poor differentiation status.

In Head & Neck Cancers (HNSCCs; oral cancer), only one paper in the literature reported that Reg3G (RegIII) levels predicted a more favorable long-term survival, whereas no difference was found with Reg3A. However, as described above, the primers designed for SYBR Green based qPCR were of low stringency and of high likelihood to form primer-dimers (self-complementarity score of 10 for Reg3A and 8 for Reg3G). Furthermore, blasting of the primer pairs revealed multiple potential targets that may be non-specifically amplified given the low stringency of the primers. A list of possible non-specifically amplified targets for Reg3G include HS3ST3A1, ANO6, EHBP1, RBM20, NECAB1, WDR3, FMN1, NECAB1, STOX1, RECK, PIAS2, CDK5RAP1, and UNC79. For Reg3A, MSL3 is a potentially non-specific target. Thus, although not previously reported in HNSCCs, the literature to date simply does not support Reg3A as a tumor-specific biomarker, either alone, or together with other candidate biomarkers.

In addition to Reg3A, hepatocyte growth factor (HGF) and its receptor, c-MET, play roles in vascular invasion, angiogenesis, metastasis and high expression levels correlate with poor patient outcomes. Binding of HGF to the c-MET receptor triggers several signal transduction pathways such as Ras/Raf/MAPK, PI3K/Akt, STAT3, and Src/focal adhesion kinase (FAK), among others. These play important roles in proliferation, survival, motility, migration, invasion, as well as angiogenesis. Notably, FAK is a key mediator of cytoplasmic reorganization and cell motility and its activity increases within minutes of HGF stimulation. As such, FAK promotes HGF-induced oral cancer migration and invasion. Moreover, FAK is a key driver and regulator of aggressive cancer spreading and metastasis, and overexpression of FAK is linked to cancer motility and is found expressed at the invasive front of tumors. In HNSCCs, particularly in oral cancers, c-MET is overexpressed in 90% of cases, with the highest expression located at sites of nodal metastasis. Paralleling c-MET expression, HGF is overexpressed in 45% of primary HNSCCs and is significantly higher in the cancers versus normal and dysplastic lesions. Therefore, identification of novel activators of the HGF-c-MET-FAK axis has important therapeutic value in HNSCCs.

Finally, in addition to HGF-c-MET-FAK signaling, E-cadherin, a Ca²⁺ dependent transmembrane glycoprotein that mediates homophilic and/or heterophilic interactions between cells via its N-terminal, is a potent tumor suppressor that maintains epithelial homeostasis and inhibits cancer cell proliferation, migration and invasion. Importantly, down-regulation, or loss of membrane-bound E-cadherin correlates with de-differentiation, high grade, as well as lymph node and distant organ metastasis in many sold tumors, including HNSCCs. Ectodomain shedding is an important post-translational process that rapidly down-regulates E-cadherin, by liberating the proteolytically cleaved ectodomain fragment (soluble E-cadherin: sEcad) into the extracellular environment and bloodstream. Prior studies have shown that sEcad, unlike intact E-cadherin, is increased in many solid tumor types, and exerts distinctly opposing functions from its membrane-bound E-cadherin counterpart by creating a permissive environment for tumor growth, survival, and invasion.

These diverse in vitro oncogenic functions of sEcad have translated clinically, such that increased serum and urine sEcad levels significantly correlate with TNM stage, tumor recurrence, and a poorer overall prognosis in many solid tumor types. An inverse relationship between membrane-bound tumoral E-cadherin and heightened levels of sEcad in the saliva of HNSCC patient's recently correlated with lymph node positivity and advanced clinical stage.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter generally relates to compositions and methods for the diagnosis and treatment of cancer that make use of and/or target Reg3A and/or Reg-related family members. In some embodiments, a method for diagnosing a cancer in a subject is provided that comprises the steps of: providing a biological sample from the subject; determining an amount of Reg3A in the sample; and comparing the amount of the Reg3A in the sample, if present, to a control level of the Reg3A. In some embodiments, the subject is then diagnosed as having cancer or a risk thereof if there is a measurable difference in the amount of the Reg3A in the sample as compared to the control level. Further, in some embodiments and as described in detailed below, the subject is then administered an effective amount of an agent capable of affecting an e xpression level or activity of Reg3A to thereby treat the cancer. In some embodiments, such an agent is an anti-Reg3A antibody that is administered alone or in combination with an effective amount of a standard cancer or chemotherapeutic agent.

With respect to the cancer capable of being diagnosed and treated in accordance with the presently-disclosed methods, in some embodiments, the cancer is a head and neck cancer, such as an oral cancer. In some embodiments, the cancer is selected from the group consisting of head and neck cancer, colorectal cancer, pancreatic cancer, ovarian cancer, cervical cancer, breast cancer, renal cell cancer, non-small cell lung cancer, small cell lung cancer, mesothelioma, gastric cancer, esophageal cancer, bladder cancer, melanoma, thyroid cancer, prostate cancer, leukemia, lymphoma, myelomas, mycoses fungoids, merkel cell cancer and/or other hematologic malignancies. In some embodiments, the cancer is within an epithelialized tissue. In some embodiments, the cancer can be characterized as a cancer of the alimentary canal, central nervous system, breast, skin, reproductive system, lung, or urinary tract. In some embodiments, such a cancer of the alimentary canal is a cancer of the mouth, throat, esophagus, stomach, intestine, rectum, or anus. In some embodiments, the cancer is a cancer of the skin, such as a squamous cell carcinoma or melanoma. In some embodiments, the cancer is a cancer of the reproductive system, such as cervical cancer, uterine cancer, ovarian cancer, vulval or labial cancer, prostate cancer, testicular cancer, or cancer of the male genital tract. In some embodiments, the subject has cancer.

Turning now to the biological samples used in accordance with the methods, in some embodiments, the biological sample comprises blood, plasma, serum, saliva, exosomes, urine, cerebrospinal fluid, ascites fluid, tissue, a tumor sample, or combinations thereof. In some embodiments, the biological sample comprises whole blood, with the whole blood including one or more immune cells, circulating tumor cells, and combinations thereof In other embodiments, the biological sample comprises exosomes or is a tumor tissue sample. In some embodiments, the biological sample is obtained prior to a treatment for the cancer.

To determine an amount of Reg3A (or sEcad and/or other Reg family members) in such biological samples, in some embodiments, determining the amount in the sample of the Reg3A or other biomarkers comprises determining the amount in the sample of the Reg3A using mass spectrometry (MS) analysis, immunoassay analysis, or both. In some embodiments, the immunoassay analysis comprises an enzyme-linked immunosorbent assay (ELISA). In some embodiments, determining the amount in the sample of the Reg3A comprises determining the amount in the sample of the Reg3A using Luminex, fluorescence-activated cell sorting (FACs), Western blot, dot blot, immunoprecipitation, immunohistochemistry, immunocytochemistry, immunofluorescence, optical spectroscopy, surface plasmon resonance, radioimmunoassay, mass spectrometry, HPLC, qPCR, RT-qPCR, multiplex qPCR, SAGE, RNA-seq, microarray analysis, fluorescence in situ hybridization (FISH), MassARRAY techniques, and combinations thereof

With that in mind, in some embodiments, determining the amount in the sample of the Reg3A (or other biomarkers) comprises determining the amount of Reg3A protein in the sample, such as by determining an amount of the Reg3A biomarker in the sample using immunohistochemistry (IHC) in a tissue sample (e.g., a tumor tissue sample) that is formalin fixed and paraffin embedded, archival, fresh, or frozen. In some embodiments, the tumor tissue sample is comprised of tumor cells, stromal cells, tumor infiltrating immune cells, and combinations thereof.

In further embodiments, determining the amount in the sample of the Reg3A (or other biomarkers) in the sample comprises determining the amount of Reg3A nucleic acid expression in the sample. For instance, in some embodiments, the nucleic acid expression is determined using qPCR, RT-qPCR, multiplex qPCR, SAGE, RNA-seq, microarray analysis, FISH, MassARRAY techniques, and combinations thereof

In some embodiments of the diagnostic methods, upon determining an expression level or activity of the Reg3A and/or other biomarkers, such as, in certain embodiments, sEcad, a treatment for the cancer is subsequently selected or modified based on the determined expression level or activity of the Reg3A or other biomarkers, such as sEcad, one or more Reg family members, and oncogenic markers. In some embodiments, a method for determining whether to initiate or continue prophylaxis or treatment of a cancer in a subject is provided that comprises the steps of: providing a series of biological samples over a time period from the subject; analyzing the series of biological samples to determine an amount in each of the biological samples of Reg3A; and comparing any measurable change in the amounts of the Reg3A in each of the biological samples to thereby determine whether to initiate or continue the prophylaxis or therapy of the cancer. In some embodiments, the series of biological samples comprises a first biological sample collected prior to initiation of the prophylaxis or treatment for the cancer and a second biological sample collected after initiation of the prophylaxis or treatment. In some embodiments, the series of biological samples comprises a first biological sample collected prior to onset of the cancer and a second biological sample collected after the onset of the cancer.

Further provided, in some embodiments of the presently-disclosed subject matter are assays and kits for diagnosing cancer in a subject. In some embodiments, an assay or process for diagnosing a cancer in a subject, comprises applying an agent capable of affecting detection of an amount of Reg3A in a biological sample obtained from the subject, and determining the amount of Reg3A in the sample. In some embodiments, a kit for diagnosing a cancer is provided that comprises an agent capable of affecting detection of an amount of Reg3A in a biological sample obtained from a subject.

Still further provided, in some embodiments of the presently-disclosed subject matter, are methods for treating a cancer. In some embodiments, a method for treating a cancer is provided that comprises administering to a subject in need thereof an effective amount of an agent capable of affecting an expression level or activity of Reg3A. In some embodiments, the agent is an anti-Reg3A antibody, such as a single chain antibody, a monoclonal antibody, or a polyclonal antibody. In some embodiments, the effective amount of the agent comprises about 1 ng/mL to about 500 μg/mL of the agent. In some embodiments, the agent capable of affecting an expression level or activity of Reg3A is administered alone or is administered in combination with an effective amount of a chemotherapeutic agent to the subject. In some embodiments, administering the agent comprises oral administration, intravenous administration, nasal or inhalation administration, intramuscular administration, intraperitoneal administration, transmucosal administration, or transdermal administration.

Further features and advantages of the present invention will become evident to those of ordinary skill in the art after a study of the description, figures, and non-limiting examples in this document.

### BRIEF DESCRIPTION OF THE DRAWINGS

FIG. 1 is a graph showing PDAC tissues expressing varying levels of Reg3A relative to Reg3G, where two PDAC tumors were homogenized and RNA extracted using the Qiagen AllPrep RNA/DNA extraction kit, and where, following cDNA synthesis, mRNA expression levels of Reg3A and Reg3G were determined using Taqman gene expression probes and expression was normalized to 18S rRNA for each tissue (Taqman assay numbers: Reg3A-Hs00170171_m1, Reg3G-Hs01595406_g1, 18S rRNA-Hs99999901_s1).

FIGS. 2A-2E includes graphs and images showing enhanced expression of Reg3A in serum, tumors, and patient-derived cells from HNSCC (oral cancer) patients, including: (FIG. 2A) a graph showing increased levels of Reg3A in the serum of cancer patients (cancer; n=57) vs. healthy controls (NL; n=29) based on a Reg3A ELISA Assay; (FIG. 2B) a graph showing Reg3A levels were significantly increased in stage II, II, and IV cancers at time of initial presentation by ELISA; (FIG. 2C) an image and graph showing immunoblot analyses of Reg3A expression in 6 resected oral tumors (T) vs. paired non-cancer tissues from the same patient (N); (FIG. 2D) images showing Reg3A immunohistochemical staining in tumor cells and CAFs from resected tumor specimens vs. normal tissues; and (FIG. 2E) immunofluorescence images showing Reg3A expression (green) in CAFs isolated from an oral cancer specimen where hoescht (blue) counterstains the nuclei. Data are presented as mean±SEM. **p<0.01; ***p<0.001 by Student's t-test.

FIG. 3 is an image showing immunohistochemical staining of Reg3A in a nerve bundle from a resected HNSCC (oral cancer) patient tumor specimen.

FIG. 4 is a graph showing that salivary Reg3A levels may predict early recurrence and/or deaths in HNSCCs (oral cancer) patients, where salivary Reg3A levels were measured by ELISA on initial presentation to the attending Oral Surgeon, where out of a total of 27 HNSCC patients (excluded one patient who died from a post-op MI), salivary Reg3A was able to be detected in 4 out of a total of 8 patient deaths (50%), and detected in 1 out of 3 recurrences (33.33%; patients still alive) and where, of note, one of the 4 patients had neck swelling (likely recurrence) and died from cardiac and pulmonary complications.

FIGS. 5A-5B includes a graph and a table showing increased Reg3A levels in the serum of HNSCC (oral cancer) patients compared to normals, including: (FIG. 5A) a graph showing Reg3A levels in the serum of 29 normal healthy controls and 59 oral cancer patients based on the Luminex xMAP multi-analyte profiling technology, where using the data Reg3A levels significantly and positively predicts the presence of HNSCCs (oral cancers), with a sensitivity of 90%, specificity of 72%, positive predictive value (PPV) of 87% and negative predictive value (NPV) of 78%.

FIGS. 6A-6B includes a graph and a table showing that Reg3A together with sEcad serves as a biomarker signature panel for HNSCCs (oral cancer), including (FIG. 6A) a graph showing sEcad serum levels in 59 HNSCC cancer patients and 30 healthy controls as measured by ELISA, where, when the sEcad ELISA was combined with the Reg3A Luminex data, the combined biomarker signature panel significantly and positively predicted the presence of HNSCCs (oral cancer), with a sensitivity of 88%, specificity of 72%, PPV of 86% and NPV of 75%, as shown in FIG. 6B.

FIGS. 7A-7D includes graphs and images showing Reg3 enhances oncogenic functions associated with tumor progression and metastasis in HNSCCs, including: (FIG. 7A) an image showing rat sciatic nerve embedded in Matrigel with/without Reg3A before addition of HNSCC cells, where Reg3A recruits cancer cells to nerve periphery (phase contrast) and also increases cancer nerve invasion (H&E); (FIG. 7B) a graph showing Reg3A-induced HNSCC cell migration and invasion in a panel of HNSCC cell lines; (FIG. 7C) images showing 3D tumor+CAF co-culture migration assays in the presence/absence of Reg3A; and (FIG. 7D) images showing a gelatin zymogram with enhanced MMP-2 and MMP-9 activity from a cancer cell (CA) and patient-derived CAF, following Reg3A stimulation.

FIGS. 8A-8G includes images and graphs showing Reg3A stimulates the HGF/c-Met axis in HNSCCs (oral cancers), including: (FIG. 8A) a graph showing qPCR screening of Reg3A targets with HGF mRNA upregulation; (FIG. 8B) an image showing Reg3A increases HGF protein expression in patient-derived CAFs; (FIG. 8C) an image showing Reg3A stimulates phosphorylated c-Met, and the downstream migration/invasion mediator FAK (pFAK) in HNSCC cells; images showing (FIG. 8D) HGF and (FIG. 8E) c-MET immunostaining in resected HNSCCs; (FIG. 8F) an image showing HGF, c-Met and Reg3A protein overexpression in HNSCC tumors compared to normal tissues; and (FIG. 8G) a graph showing quantification of (FIG. 8F) with n=6 patients. Data are represented as mean±SEM. **p<0.01 by Student's t-test.

FIGS. 9A-9B includes images showing that Reg3A partially co-localizes with c-MET in HNSCC tumors, including: (FIG. 9A) an image showing double immunofluorescence staining for Reg3A (green) and c-MET (red) demonstrating overlap (yellow) in select patient tumors; and (FIG. 9B) an image showing double immunofluorescence staining for Reg3A (green) and c-MET (red) demonstrating overlap (yellow) in an oral cancer cell line, with Hoechst staining of the nuclei blue.

FIGS. 10A-10D includes graphs showing that an anti-Reg3A antibody decreases HNSCC (oral cancer) and PDAC cell viability, including: (FIG. 10A) a graph showing SCC15 cell viability (CellTiter-Glo, Promega) was reduced following anti-Reg3A mAb administration (*p<0.05 vs. Control); and (FIGS. 10B-10D) graphs showing anti-Reg3A mAb either alone, or together with Gemcitabine (0.2 μM), Cisplatin (5 μg/ml) or Paclitaxel (0.5 μM) decreases PDAC cell viability (Promega), *p<0.05, **p<0.01, ***p<0. 001 as compared to corresponding IgG group.

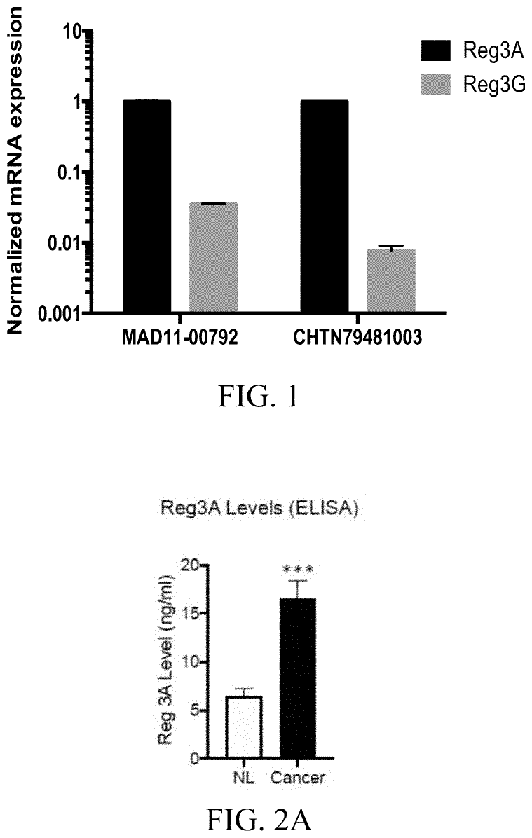

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter includes compositions, systems, and methods that target Reg3A or make use of Reg3A as a biomarker, either alone or in combination with other oncogenic biomarkers, for the treatment, diagnosis, and prognosis of cancer. In some embodiments, a method of diagnosing a cancer is provided that comprises the steps of obtaining a biological sample from a subject, and determining an amount in the sample of Reg3A present in the biological sample. In some embodiments, the Reg3A is the Reg3A identified as having UniProt Accession Number Q06141. In some embodiments, the amount of Reg3A is determined either alone and/or in combination with the determination of an amount of other Reg family members, including, but not limited to the Reg family members identified in Table 1 below (e.g., RegIA (Reg1A), RegIB (Reg1B), RegIIIA (Reg3A, HIP/HAP), RegIII (Reg3G) and RegIV (Reg4).

TABLE 1
Gene symbols, mRNA and protein accession numbers, and alternative names for
REG protein family members. Table contains the current database annotations
for human REG protein family members along with alternative names used in the literature.

| Human | | | | | |
|---|---|---|---|---|---|
| Reg | NCBI | | Ref Seq | UniProt | |
| Gene | Gene | Splice | Accession | Accession | |
| Symbol | ID | Variants | Numbers | Number | Alternative Names |
| REG1A | 5967 | N/A | NM_002909 | P05451 | Lithostathine-1-alpha |
| | | | | | Islet cells regeneration factor (ICRF) |
| | | | | | Islet of Langerhans regenerating protein (REG) |
| | | | | | Pancreatic stone protein (PSP) |
| | | | | | Pancreatic thread protein (PTP) |
| | | | | | Regenerating islet-derived protein 1-alpha (REG-1-alpha) |
| | | | | | Regenerating protein 1 alpha |
| REG1B | 5968 | N/A | NM_006507 | P48304 | Lithostathine-1-beta |
| | | | | | Pancreatic stone protein 2 (PSP-2) |
| | | | | | Regenerating islet-derived protein 1-beta (REG-1-beta) |
| | | | | | Regenerating protein I beta |
| REG3A | 5068 | Variant 1 | NM_002580 | Q06141 | Regenerating islet-derived protein 3-alpha |
| | | Variant 2 | NM_138937 | | Hepatointestinal pancreatic protein (HIP/PAP) |
| | | Variant 3 | NM_138938 | | Pancreatitis-associated protein 1 (PAP1) |
| | | | | | Regenerating islet-derived protein III-alpha (Reg III alpha) |
| | | | | | Islet neogenesis associated protein (INGAOP) |
| REG3G | 130120 | Variant 1 | NM_001008387 | Q6UW15-1 | Regenerating islet-derived protein 3-gamma |
| | | Variant 2 | NM_198448 | Q6UW15-1 | Pancreatitis-associated protein 1B (PAP-1B, PAP1B, PAPIB) |
| | | Variant 3 | NM_001270040 | Q6UW15-2 | Regenerating islet-derived protein III-gamma (REGIII) |
| REG4 | 83998 | Variant 1 | NM_001159352 | Q9BYZ8-1 | Regenerating islet-derived protein 4 (REG-4) |
| | | Variant 2 | NM_032044 | Q9BYZ8-1 | Gastrointestinal secretory protein (GISP) |
| | | Variant 3 | NM_001159353 | Q9BYZ8-2 | REG-like protein (RELP) |
| | | | | | Regenerating islet-derived protein IV (Reg IV) |

In some embodiments, a method for diagnosis or prognosis of a cancer in a subject is provided that comprises the steps of: obtaining a biological sample; determining an amount of Reg3A present in the biological sample; and comparing the amount of the Reg3A in the sample, if present, to a control level of the Reg3A, wherein the subject is diagnosed as having a cancer or a risk thereof if there is an increase in the amount of the Reg3A in the sample as compared to the control level. In some embodiments, the presently-disclosed subject matter includes methods and systems for diagnosing cancer a subject, and for determining whether to initiate or continue prophylaxis or treatment of cancer in a subject, by determining an amount of Reg3A alone or in combination with other biomarkers in a biological sample from a subject. In some embodiments, the Reg3A biomarkers are determined in combination with the other Reg family members described above and/or with soluble E-cadherin as described in further detail below.

The exemplary human biomarkers described herein are not intended to limit the present subject matter to human polypeptide biomarkers or mRNA biomarkers only. Rather, the present subject matter encompasses biomarkers across animal species that are associated with cancer. In addition, standard gene/protein nomenclature guidelines generally stipulate human gene name abbreviations are capitalized and italicized and protein name abbreviations are capitalized, but not italicized. Further, standard gene/protein nomenclature guidelines generally stipulate mouse, rat, and chicken gene name abbreviations italicized with the first letter only capitalized and protein name abbreviations capitalized, but not italicized. In contrast, the gene/protein nomenclature used herein when referencing specific biomarkers uses all capital letters for the biomarker abbreviation, but is intended to be inclusive of genes (including mRNAs and cDNAs) and proteins across animal species.

A “biomarker” is a molecule useful as an indicator of a biologic state in a subject. With reference to the present subject matter, the biomarkers disclosed herein can be polypeptides that exhibit a change in expression or state, which can be correlated with the risk of developing, the presence of, or the progression of cancer in a subject. In addition, the biomarkers disclosed herein are inclusive of messenger RNAs (mRNAs) encoding the biomarker polypeptides, as measurement of a change in expression of an mRNA can be correlated with changes in expression of the polypeptide encoded by the mRNA. As such, determining an amount of a biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

The terms “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example an amount of Reg3A, the amount (including presence or absence) of which is indicative of the presence, severity, or absence of the condition.

Along with diagnosis, clinical disease prognosis is also an area of great concern and interest. It is important to know the stage and rapidity of advancement of the cancer in order to plan the most effective therapy. If a more accurate prognosis can be made, appropriate therapy, and in some instances less severe therapy for the patient can be chosen. Measurement of Reg3A and/or other biomarker levels disclosed herein can be useful in order to categorize subjects according to advancement of the cancer who will benefit from particular therapies and differentiate from other subjects where alternative or additional therapies can be more appropriate.

As such, “making a diagnosis” or “diagnosing”, as used herein, is further inclusive of determining a prognosis, which can provide for predicting a clinical outcome (with or without medical treatment), selecting an appropriate treatment (or whether treatment would be effective), or monitoring a current treatment and potentially changing the treatment, based on the measure of diagnostic biomarker levels disclosed herein (e.g., the amount of Reg3A).

The phrase “determining a prognosis” as used herein refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the presence, absence or levels of test biomarkers. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not having an increase in Reg3A), the chance of a given outcome may be about 3%. In certain embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome is a statistical analysis. For example, a Reg3A level of greater than a control level in some embodiments can signal that a subject is more likely to suffer from a cancer than subjects with a Reg3A level less than or equal to the control level, as determined by a level of statistical significance. Additionally, a change in Reg3A levels from baseline levels can be reflective of subject prognosis, and the degree of change in Reg3A levels can be related to the severity of adverse events. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In other embodiments, a threshold degree of change in the level of a prognostic or diagnostic indicator can be established, and the degree of change in the level of the indicator in a biological sample can simply be compared to the threshold degree of change in the level. A preferred threshold change in the level of Reg3A described herein is about 5%, about 10%, about 15%, about 20%, about 25%, about 30%, about 50%, about 75%, about 100%, and about 150%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments of the presently-disclosed subject matter, multiple determination of one or more diagnostic or prognostic indicators can be made, and a temporal change in the amount of Reg3A and/or other biomarkers can be used to monitor the progression of disease and/or efficacy of appropriate therapies directed against the disease. In such an embodiment, for example, one might expect to see a decrease in Reg3A levels over time during the course of effective therapy. Thus, the presently-disclosed subject matter provides in some embodiments a method for determining treatment efficacy and/or progression of a cancer in a subject. In some embodiments, the method comprises determining an amount of Reg3A in biological samples collected from the subject at a plurality of different time points and comparing the amounts of Reg3A in the samples collected at different time points. For example, a first time point can be selected prior to initiation of a treatment and a second time point can be selected at some time after initiation of the treatment. One or more Reg3A levels can then be measured in each of the samples taken from different time points and qualitative and/or quantitative differences noted. A change in the amounts of the biomarker levels from the first and second samples can be correlated with determining treatment efficacy and/or progression of the disease in the subject. In some embodiments, such one or more Reg3A levels can be measured alone or, in other embodiments, such Reg3A levels can be measured at multiple time points along with other biomarkers, such as other Reg family members or soluble E-cadherin.

The terms “correlated” and “correlating,” as used herein in reference to the use of diagnostic and prognostic biomarkers, refers to comparing the presence or quantity of the biomarkers in a subject to its presence or quantity in subjects known to suffer from, or known to be at risk of, a given condition (e.g., a cancer); or in subjects known to be free of a given condition, i.e. “normal individuals”. For example, a Reg3A level in a biological sample can be compared to a level known to be associated with a specific type of cancer. The sample's Reg3A level is said to have been correlated with a diagnosis; that is, the skilled artisan can use the Reg3A level to determine whether the subject suffers from a specific type of cancer, and respond accordingly. Alternatively, the sample's Reg3A level can be compared to a control marker level known to be associated with a good outcome (e.g., the absence of a cancer), such as an average level found in a population of normal subjects.

In certain embodiments, a diagnostic or prognostic biomarker is correlated to a condition or disease by merely its presence or absence. In other embodiments, a threshold level of a diagnostic or prognostic Reg3A amount can be established, and the level of the Reg3A in a subject sample can simply be compared to the threshold level.

As noted, in some embodiments, multiple determination of one or more diagnostic or prognostic biomarkers can be made, and a temporal change in the marker can be used to determine a diagnosis or prognosis. For example, a diagnostic level of Reg3A can be determined at an initial time, and again at a second time. In such embodiments, an increase in the Reg3A levels from the initial time to the second time can be diagnostic of a particular type of cancer or a given prognosis. Furthermore, the degree of change of one or more markers can be related to the severity of cancer and future adverse events, including metastasis.

The skilled artisan will understand that, while in certain embodiments comparative measurements can be made of the same diagnostic marker at multiple time points, one can also measure a given marker at one time point, and a second marker at a second time point, and a comparison of these markers can provide diagnostic information.

With regard to the step of providing a biological sample from the subject, the term “biological sample” as used herein refers to any body fluid or tissue potentially comprising a Reg3A and/or the other biomarkers described herein. In some embodiments, for example, the biological sample comprises plasma, serum, urine, saliva, peripheral blood mononuclear cells (PBMCS), cerebrospinal fluid, exosomes, circulating tumor cells, cerebrospinal fluid, ascites fluid, tissue, a tumor sample, and combinations, or sub-fractions thereof. In some embodiments, the sample is whole blood. In some embodiments, the whole blood comprises one or more immune cells, circulating tumor cells and any combinations thereof. In some embodiments, the biological samples comprises exosomes. In some embodiments, the Reg3A biomarker or biomarker panel comprises one or more cells obtained from a tumor biopsy or other sample, the cells of which may include cancer cells, stromal cells, or immune cells, or any combination thereof, obtained from a tumor biopsy or other source. In some embodiments, the sample is a tumor tissue sample. In some embodiments, the tissue sample is formalin fixed and paraffin embedded, archival, fresh or frozen.

Turning now to the step of identifying an amount of Reg3A present in the biological sample, various methods known to those skilled in the art can be used to identify such Reg3A or other biomarker levels in the provided biological sample. In some embodiments, determining the amount of biomarkers in samples comprises using a RNA measuring assay to measure mRNA encoding biomarker polypeptides in the sample and/or using a protein measuring assay to measure amounts of biomarker polypeptides in the sample.

In certain embodiments, the amounts of biomarkers can be determined by probing for mRNA of the biomarker in the sample using any RNA identification assay known to those skilled in the art. Briefly, RNA can be extracted from the sample, amplified, converted to cDNA, labeled, and allowed to hybridize with probes of a known sequence, such as known RNA hybridization probes (selective for mRNAs encoding biomarker polypeptides) immobilized on a substrate, e.g., array, or microarray, or quantitated by real time PCR (e.g., quantitative real-time PCR, such as available from Bio-Rad Laboratories, Hercules, Calif., U.S.A.). Because the probes to which the nucleic acid molecules of the sample are bound are known, the molecules in the sample can be identified. In this regard, DNA probes for one or more biomarkers (e.g., Reg3A) can be immobilized on a substrate and provided for use in practicing a method in accordance with the present subject matter.

With regard to determining amounts of biomarker polypeptides in samples, mass spectrometry and/or immunoassay devices and methods can be used to measure biomarker polypeptides in samples, although other methods are well known to those skilled in the art as well. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Thus, in certain embodiments of the presently-disclosed subject matter, the marker peptides are analyzed using an immunoassay. The presence or amount of a marker (e.g., Reg3A) can be determined using antibodies or fragments thereof specific for each marker and detecting specific binding. For example, in some embodiments, the antibody specifically binds Reg3A, which is inclusive of antibodies that bind the full-length peptide or a fragment thereof In some embodiments, the antibody is a monoclonal antibody, such as the Reg3A monoclonal antibody described herein below as being useful for the treatment of cancer. In other embodiments, the antibody is a polyclonal antibody.

Any suitable immunoassay can be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies or fragments thereof specific for the markers is also contemplated by the presently-disclosed subject matter. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

In some embodiments, mass spectrometry (MS) analysis can be used alone or in combination with other methods (e.g., immunoassays) to determine the presence and/or quantity of the one or more biomarkers of interest (e.g., Reg3A) in a biological sample. In some embodiments, the MS analysis comprises matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers, such as for example triple quadrupole mass spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated herein by this reference.

With further respect to the measurement of the biomarkers described herein, in some embodiments, the Reg3A biomarker or biomarker panel is detected in the sample using a method selected from the group consisting of ELISA, Luminex, FACs, Western blot, dot blot, immunoprecipitation, immunohistochemistry, immunocytochemistry, immunofluorescence, immunodetection methods, optical spectroscopy, radioimmunoassay, mass spectrometry, HPLC, qPCR, RT-qPCR, multiplex qPCR, SAGE, RNA-seq, microarray analysis, FISH, MassARRAY technique, and combinations thereof.

In some embodiments, the Reg3A biomarker or biomarker panel is detected in the sample by protein expression. In some embodiments, protein expression is determined by immunohistochemistry, immunocytochemistry or immunofluorescence. In some embodiments, the Reg3A biomarker or biomarker panel is detected on tumor cells, tumor infiltrating cells, stromal cells and any combination thereof In some embodiments, the staining is membrane, cytosolic, nuclear or combinations thereof.

Although certain embodiments of the method only call for a qualitative assessment of the presence or absence of the one or more markers in the biological sample, other embodiments of the method call for a quantitative assessment of the amount of each of the one or more markers in the biological sample. Such quantitative assessments can be made, for example, using one of the above mentioned methods, as will be understood by those skilled in the art.

As mentioned above, depending on the embodiment of the method, identification of the amount of Reg3A or other markers can be a qualitative determination of the presence or absence of the markers, or it can be a quantitative determination of the concentration of the markers. In this regard, in some embodiments, the step of identifying the subject as having cancer or a risk thereof requires that certain threshold measurements are made, i.e., the levels of Reg3A in the biological sample are above a control level. In certain embodiments of the method, the control level is any detectable level of the Reg3A or other markers. In other embodiments of the method where a control sample is tested concurrently with the biological sample, the control level is the level of detection in the control sample. In other embodiments of the method, the control level is based upon and/or identified by a standard curve. In other embodiments of the method, the control level is a specifically identified concentration, or concentration range. As such, the control level can be chosen, within acceptable limits that will be apparent to those skilled in the art, based in part on the embodiment of the method being practiced and the desired specificity, etc.

In some embodiments of the presently-disclosed subject matter, it is contemplated that the efficacy, accuracy, sensitivity, and/or specificity of the method can be enhanced by probing for multiple markers in the biological sample. For example, in certain embodiments of the method, the biological sample can be probed for Reg3A in combination with other Reg family members such as those described herein above or with soluble E-cadherin (sEcad; see, e.g., UniProt Identification No. P12830, which provides the UniProt ID E-cadherin full sequence with soluble E-cadherin or sEcad being found from 155-707 aa).

With respect to the cancer diagnosed in accordance with the presently-disclosed subject matter, the term “cancer” is used herein to refer to all types of cancer or neoplasm or malignant tumors found in animals, including leukemias, carcinomas, melanoma, and sarcomas. In some embodiments, the cancer or malignancy is selected from brain, medulloblastoma, breast, lung, non-small cell lung, mesothelioma, GI tract (e.g. esophagus, stomach, intestine, colon etc.), pancreas, bladder, ovary, prostate, skin (e.g. melanoma, cutaneous SCCs, basal cell carcinomas etc.), uterus, other epithelial cancers, cancers of tissues derived from the ectoderm (e.g. central nervous system, lens of the eye, cranial and sensory ganglia and nerves, and connective tissue in the head), sarcomas, thyroid cancers, leukemia, lymphomas and other hematologic malignancies. In some embodiments, the cancer is a head and neck cancer, such as, in certain embodiments, an oral cancer.

In some embodiments, the cancer is selected from the group consisting of head and neck cancer (e.g., oral cancer), colorectal cancer, pancreatic cancer, ovarian cancer, cervical cancer, breast cancer, renal cell cancer, non-small cell lung cancer, small cell lung cancer, mesothelioma, gastric cancer, esophageal cancer, bladder cancer, melanoma, thyroid cancer, prostate cancer, leukemia, lymphoma, myelomas, mycoses fungoids, merkel cell cancer and/or other hematologic malignancies. In some embodiments, the cancer is within an epithelialized tissue. In some embodiments, the cancer is a cancer of the alimentary canal, central nervous system, breast, skin, reproductive system, lung, or urinary tract. In some embodiments, the cancer of the alimentary canal is a cancer of the mouth, throat, esophagus, stomach, intestine, rectum, or anus. In some embodiments, the cancer of the skin is squamous cell carcinoma or melanoma. In some embodiments, the cancer of the reproductive system is cervical cancer, uterine cancer, ovarian cancer, vulval or labial cancer, prostate cancer, testicular cancer, or cancer of the male genital tract.

With respect to the cancers describes herein, by “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some embodiments of the presently-disclosed subject matter, a system, kit, or assay for detecting Reg3A and/or other biomarkers and/or for determining an amount of Reg3A and/or other biomarkers is provided. Such systems, kits, and assays can be provided, for example, as commercial kits that can be used to test a biological sample, or series of biological samples, from a subject. The system can also include certain samples for use as controls. The system can further include one or more standard curves providing levels of markers as a function of assay units.

In some embodiments, an assay for diagnosing a cancer in a subject is provided that comprises the steps of: applying an agent capable of affecting detection of an amount of Reg3A in a biological sample obtained from the subject; and determining the amount of Reg3A in the sample.

In some embodiments, a system, kit, or assay for the analysis of biomarkers is provided that comprises an agent capable of affecting detection of an amount of Reg3A in a biological sample obtained from a subject, such as antibodies having specificity for Reg3A. Such a system, kit, or assay can comprise devices and reagents for the analysis of at least one test sample. The system can further comprise instructions for using the system and conducting the analysis on a sample obtained from a subject.

Still further provided in some embodiments of the presently-disclosed subject matter are therapeutic methods for treating a cancer. In some embodiments, a method for treating a cancer is provided that comprises administering to a subject in need thereof an effective amount of an agent capable of affecting an expression level or activity of Reg3A. In some embodiments, the agent capable of affecting an expression level or activity of Reg3A is an anti-Reg3A antibody, such as the commercially-available human Reg3A monoclonal antibody produced by R&D Systems, Cat # MAB5965 (see also E. coli-derived recombinant human Reg3A; Glu27-Asp175; Accession # Q06141). In some embodiments, the anti-Reg3A antibody can be a single chain antibody, or a monoclonal or polyclonal antibody. Regardless of the source of the antibody, however, suitable antibodies can include intact antibodies, for example, IgG tetramers having two heavy (H) chains and two light (L) chains, single chain antibodies, chimeric antibodies, humanized antibodies, complementary determining region (CDR)-grafted antibodies as well as antibody fragments, e.g., Fab, Fab′, F(ab′)2, scFv, Fv, and recombinant antibodies derived from such fragments, e.g., camelbodies, microantibodies, diabodies, nanobodies, and bispecific antibodies.

An intact antibody is one that comprises an antigen-binding variable region (VH and VL) as well as a light chain constant domain (CL) and heavy chain constant domains, CHL CH2 and CH3. The constant domains may be native sequence constant domains (e.g. human native sequence constant domains) or amino acid sequence variants thereof. As is well known in the art, the VH and VL regions are further subdivided into regions of hypervariability, termed “complementarity determining regions” (CDRs), interspersed with the more conserved framework regions (FRs). The extent of the FRs and CDRs has been defined (see, Kabat et al. Sequences of Proteins of Immunological Interest, Fifth Edition, U.S. Department of Health and Human Services, NIH Publication No. 91-3242, 1991, and Chothia, et al., J. Mol. Biol. 196:901-917 (1987). The CDR of an antibody typically includes amino acid sequences that together define the binding affinity and specificity of the natural Fv region of a native immunoglobulin binding site.

An anti-Reg3A antibody can be from any class of immunoglobulin, for example, IgA, IgG, IgE, IgD, IgM (as well as subtypes thereof (e.g., IgG1, IgG2, IgG3, and IgG4)), and the light chains of the immunoglobulin may be of types kappa or lambda. The recognized human immunoglobulin genes include the kappa, lambda, alpha (IgA1 and IgA2), gamma (IgG1, IgG2, IgG3, IgG4), delta, epsilon, and mu constant region genes, as well as the myriad immunoglobulin variable region genes.

As noted, other useful antibody formats include diabodies, minibodies and bispecific antibodies. A diabody is a homodimer of scFvs that are covalently linked by a short peptide linker (about 5 amino acids or less). By using a linker that is too short to allow pairing between two domains on the same chain, the domains can be forced to pair with the complementary domains of another chain and create two antigen-binding sites (see, e.g., EP 404,097 and WO 93/11161 for additional information regarding diabodies).

A bispecific antibody, which recognizes two different epitopes, can also be used as long as one arm specifically binds Reg3A, sEcad or another protein of interest of the presently-disclosed subject matter, as described herein. A variety of different bispecific antibody formats have been developed. For example, useful bispecific antibodies can be quadromas, i.e., an intact antibody in which each H-L pair is derived from a different antibody. Typically, quadromas are produced by fusion of two different B cell hybridomas, followed by screening of the fused calls to select those that have maintained the expression of both sets of clonotype immunoglobulin genes. Alternatively, a bispecific antibody can be a recombinant antibody. Exemplary formats for bispecific antibodies include, but are not limited to tandem scFvs in which two single chains of different specificity are connected via a peptide linker; diabodies and single chain diabodies.

In some embodiments, such anti-Reg3A or other antibodies can be administered alone and/or in combination with chemotherapeutic agents, radiation, and/or other cancer agents known to those skilled in the art, including immune checkpoint inhibitors, target therapies, anti-HGF/c-MET pathway axis drugs, and the like. In some embodiments, the Reg3A biomarker or biomarker panels described herein above are used for selecting the most appropriate therapy for the subject having the cancer, whereby the presence or amount of the biomarker or biomarkers in a particular biological sample from the subject can be used to provide a determination of the recommended course of therapy for the subject. For example, in some embodiments, the therapeutic methods described herein further comprise the step of providing a biological sample from the subject and determining an amount of Reg3A, a Reg family member, and/or sEcad in the biological sample, where the effective amount administered to the subject is based on the amount of Reg3A, the Reg family member, and/or the sEcad in the biological sample. In some embodiments, the biological samples utilized in such therapeutic methods comprise urine, saliva, cerebrospinal fluid, blood, exosomes, or a tumor sample.

In some embodiments, compositions comprising two or more agents that specifically target one or more of the regions of Reg3A may be administered to persons or mammals suffering from, or predisposed to suffer from, cancer. Such anti-Reg3A agents may also be administered with another therapeutic agent, such as a cytotoxic agent, or cancer chemotherapeutic agent. Of course, concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

Exemplary cytotoxic agents include radioactive isotopes (e.g., ¹³¹I, ¹³⁵I, ⁹⁰Y and 186Re) and toxins such as enzymatically active toxins of bacterial, fungal, plant or animal origin or synthetic toxins, or fragments thereof. In some embodiments, such cytotoxic agents are administered along with a non-cytotoxic agent. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with an antibody or other agent disclosed herein.

As noted, a number of conventional cancer medicaments can be administered in accordance with the methods disclosed herein (i.e., in conjunction with an anti-Reg3A agent). Such useful medicaments include, in certain embodiments, anti-angiogenic agents, i.e., agents that block the ability of tumors to stimulate new blood vessel growth necessary for their survival. Any anti-angiogenic agent known to those in the art can be used, including agents such as Bevacizumab (Avastin®, Genentech, Inc.) that block the function of vascular endothelial growth factor (VEGF). Other examples include, without limitation, Dalteparin (Fragmin®), Suramin ABT-510, Combretastatin A4 Phosphate, Lenalidomide, LY317615(Enzastaurin), Soy Isoflavone (Genistein; Soy Protein Isolate) AMG-706, Anti-VEGF antibody, AZD2171, Bay 43-9006 (Sorafenib tosylate), PI-88, PTK787/ZK 222584 (Vatalanib), SU11248 (Sunitinib malate), VEGF-Trap, XL184, ZD6474, Thalidomide, ATN-161, EMD 121974 (Cilenigtide) and Celecoxib (Celebrex®).

Other useful therapeutics include those agents that promote DNA-damage, e.g., double stranded breaks in cellular DNA, in cancer cells. Any form of DNA-damaging agent know to those of skill in the art can be used. DNA damage can typically be produced by radiation therapy and/or chemotherapy. Examples of radiation therapy include, without limitation, external radiation therapy and internal radiation therapy (also called brachytherapy). Energy sources for external radiation therapy include x-rays, gamma rays and particle beams; energy sources used in internal radiation include radioactive iodine (iodine¹²⁵ or iodine¹³¹), and from strontium⁸⁹, or radioisotopes of phosphorous, palladium, cesium, iridium, phosphate, or cobalt. Methods of administering radiation therapy are well known to those of skill in the art. Examples of such DNA-damaging chemotherapeutic agents include, without limitation, Busulfan (Myleran), Carboplatin (Paraplatin), Carmustine (BCNU), Chlorambucil (Leukeran), Cisplatin (Platinol), Cyclophosphamide (Cytoxan, Neosar), Dacarbazine (DTIC-Dome), Ifosfamide (Ifex), Lomustine (CCNU), Mechlorethamine (nitrogen mustard, Mustargen), Melphalan (Alkeran), and Procarbazine (Matulane).

In some embodiments, other standard cancer chemotherapeutic agents can also be utilized including, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar®); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol®), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin®), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, carminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex®), interferon-β, mepitiostane, mitobronitol, melphalan, laminin peptides, lentinan, Coriolus versicolor extract, tegafur/uracil, estramustine (estrogen/mechlorethamine).

Additional agents which may be used as therapy for cancer patients include EPO, G-CSF, ganciclovir; antibiotics, leuprolide; meperidine; zidovudine (AZT); interleukins 1 through 18, including mutants and analogues; interferons or cytokines, such as interferons α, β, and γ hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), c-Met and insulin growth factor (IGF); tumor necrosis factor-α& β (TNF-α& β); invasion inhibiting factor-2 (IIF-2); bone morphogenetic proteins 1-7 (BMP 1-7); somatostatin; thymosin-α-1; γ-globulin; superoxide dismutase (SOD); complement factors; and anti-angiogenesis factors.

With respect to the treatment of the cancer, as used herein, the terms “treatment” or “treating” relate to any treatment of a cancer, including, but not limited to, prophylactic treatment and therapeutic treatment. As such, the terms treatment or treating include, but are not limited to: reducing an amount of a cancer or the development of a cancer; inhibiting the progression of a cancer; arresting or preventing the development of a cancer; reducing the severity of a cancer; ameliorating or relieving symptoms associated with a cancer; and causing a regression of the cancer or one or more of the symptoms associated with the cancer. In some embodiments, the cancer is a head and neck cancer, such as, in some embodiments, an oral cancer. In some embodiments, the cancer is within an epithelialized tissue. In some embodiments, the cancer is a cancer of the alimentary canal, central nervous system, breast, skin, reproductive system, lung, or urinary tract. In some embodiments, the cancer of the alimentary canal is a cancer of the mouth, throat, esophagus, stomach, intestine, rectum, or anus. In some embodiments, the cancer of the skin is squamous cell carcinoma or melanoma. In some embodiments, the cancer of the reproductive system is cervical cancer, uterine cancer, ovarian cancer, vulval or labial cancer, prostate cancer, testicular cancer, or cancer of the male genital tract.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m2. In some embodiments, the effective amount of an agent capable of affecting expression or activity of Reg3A is administered in an amount of about 1 ng/mL to about 500 μg/mL.

Suitable methods for administering a therapeutic composition in accordance with the methods of the presently-disclosed subject matter include, but are not limited to, systemic administration, parenteral administration (including intravascular, intramuscular, intraarterial administration), oral delivery, topical administration, buccal delivery, rectal delivery, vaginal delivery, subcutaneous administration, intraperitoneal administration, inhalation, intratracheal installation, surgical implantation, transdermal delivery, local injection, and hyper-velocity injection/bombardment. In some embodiments, administering the agent capable of affecting an expression or activity level of Reg3A comprises oral administration, intravenous administration, nasal or inhalation administration, intramuscular administration, intraperitoneal administration, transmucosal administration, or transdermal administration.

Regardless of the route of administration or particular use of a composition of the presently-disclosed subject matter, the compositions are typically administered or otherwise used in an amount effective to achieve the desired response. As used herein, the terms “effective amount” and “therapeutically effective amount” refer to an amount of the therapeutic composition sufficient to produce a measurable biological response (e.g., a reduction in cancer cells). Actual dosage levels of active ingredients in a therapeutic composition of the presently-disclosed subject matter can be varied so as to administer or make use of an amount of the active composition that is effective to achieve the desired response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered or used, and the dose is escalated in the absence of dose-limiting toxicity to a minimally effective amount. Determination and adjustment of an effective dose, as well as evaluation of when and how to make such adjustments, are known to those of ordinary skill in the art.

For additional guidance regarding formulation and dose, see U.S. Pat. Nos. 5,326,902 and 5,234,933; PCT International Publication No. WO 93/25521; Berkow, et al., (1997) The Merck Manual of Medical Information, Home ed. Merck Research Laboratories, Whitehouse Station, N.J.; Goodman, et al., (2006) Goodman & Gilman's the Pharmacological Basis of Therapeutics, 11th ed. McGraw-Hill Health Professions Division, N.Y.; Ebadi. (1998) CRC Desk Reference of Clinical Pharmacology. CRC Press, Boca Raton, Fla.; Katzung, (2007) Basic & Clinical Pharmacology, 10th ed. Lange Medical Books/McGraw-Hill Medical Pub. Division, New York; Remington, et al., (1990) Remington's Pharmaceutical Sciences, 18th ed. Mack Pub. Co., Easton, Pa.; Speight, et al., (1997) Avery's Drug Treatment: A Guide to the Properties, Choice, Therapeutic Use and Economic Value of Drugs in Disease Management, 4th ed. Adis International, Auckland/ Philadelphia; and Duch, et al., (1998) Toxicol. Lett. 100-101:255-263, each of which are incorporated herein by reference.

With respect to the presently-disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently-disclosed subject matter. As such, the presently-disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples.

### EXAMPLES

### Materials and Methods for Examples 1-4

Cell culture and reagents. Human Ca127, SCC4, Fadu, HPAF II and BxPC3 cells were purchased from American Type Culture Collection [ATCC; authentication by short tandem repeat (STR) profiling/isoenzyme analysis/karyotyping), and maintained in the suggested ATCC culture media, unless otherwise specified. All cultures were maintained at 37° C. in a humidified atmosphere containing 5% CO₂. Recombinant human Reg3A (rhReg3A) and the anti-Reg3A antibody were purchased from R&D systems.

Western blotting. Tissue or cells were lysed using lysis buffer (50 mM Tris pH7.5, 150 mM NaCl, 5 mM NaF, 1% NP40, 0.5% sodium deoxycholate, 0.1% SDS, 20 mM sodium pyrophosphate, 40 mM beta-glycerophospate, 1 mM Na3VO₄, and 1 mM PMSF) or in RIPA buffer containing protease inhibitor cocktail (Sigma) for 30 min on ice. Lysates were centrifuged at 10,000 rpm, supernatants collected, and protein concentration measured using the BCA Protein Assay Kit (Pierce). Samples were denatured in sample buffer (10% glycerol, 2% SDS, bromophenol blue and 2.5% β-mercaptoethanol) and heated to 95-100° C. for 5 min before gel electrophoresis. Samples were separated by SDS-PAGE, transferred to nitrocellulose membranes, blocked with 5% (w/v) nonfat dry milk in TBST for 1 hr to block nonspecific binding, and incubated with primary antibodies at 4° C. overnight. Proteins were detected using appropriate HRP-conjugated secondary antibodies in blocking buffer. Blots were developed using an enhanced chemiluminescence detection kit (Santa Cruz). Western blot signals were quantitated by Image' Alternatively, Bio-Rad TGX Stain-Free precast gels were used, and bands analyzed using the ChemiDoc MP Imaging System (Bio-Rad). Some of the immunoblots were reprobed after stripping (Restore Western Blot stripping buffer; Thermo Scientific). The following antibodies were used: Reg3A (Invitrogen, CA), p-MET (Cell Signaling), c-MET (Cell Signaling), pFAK (Cell Signaling), HGF (Protein Tech), and actin (Santa Cruz).

Histopathology and immunohistochemistry. Tissues were fixed in 10% formalin, embedded in paraffin and 5 μm sections were prepared and stained with hematoxylin and eosin (H&E). IHC was performed as previously described (Brouxhon et al., 2013). Briefly, after xylene de-paraffinization and ethanol dehydration, sections underwent heat-antigen retrieval with citrate buffer (Dako, CA) for 90 min, as per the manufacturer's instructions. Endogenous peroxidases were blocked by 3% H₂O₂/H₂O for 10 minutes, and nonspecific binding prevented by incubation in normal goat serum for 30 minutes at room temperature (RT). Slides were incubated in the Reg3A primary antibody (Thermo Fisher Scientific) diluted in PBS with 5% goat serum overnight at 4° C. Sections were then incubated with a biotinylated anti-rabbit secondary antibody conjugated with HRP (Jackson Immunoresearch) for 1 hr at RT. Bound peroxidases were visualized by incubation in a 3-3′ diaminobenzidine (DAB) solution for 1-20 min (Vector Laboratories), and staining carried out using the Vectastain ABC Kit (Vector Laboratories, CA). Sections were washed, counterstained with hematoxylin, dehydrated, and mounted. The images were acquired with a BX51 Olympus light microscope, and quantification performed using ImageJ or Image Pro Plus on 20× magnification images.

Immunofluorescence. Cells were cultured on chamber slides (no. 177437; Nalge Nunc International), fixed with 10% formalin or 100% methanol, blocked in PBS containing 10% normal goat serum (NGS), and incubated for 1 hr with an anti-Reg3A antibody (Thermo Fischer Scientific) containing 10% NGS and 0.4% Triton X-100. Cells were washed with PBS, incubated with secondary antibodies conjugated to Alexa-488 or Alexa-594 (Life Technologies) for 1.5 hr and mounted with ProLong (Thermo Fisher). Double IF staining for Reg3A (Sinobiological) and c-MET (Cell Signaling Technology) was similar to IHC, but performed by respectively incubating tissues with secondary antibodies conjugated to Alexa-488 or Alexa-594 (Life Technologies), and subsequent incubation with monovalent Fab fragments (Jackson ImmunoResearch). Slides were counter-stained with DAPI/anti-fade mounting medium (Vector Laboratories), and examined by fluorescence microscopy.

Migration and invasion assays. For migration and invasion assays, tumor cells were seeded in the upper compartment of 8.0 μm pore size Control Insert and Matrigel Invasion Chamber 24-well plates (BD Bioscience), respectively. Upper chambers were cultured with DMEM/F12 media, while the lower compartment was filled with DMEM/F12 media containing PBS or Reg3A (400 μg/ml) in the presence or absence of 0.4% FBS. For co-culture migration assays, patient-derived CAFs were added to the bottom layer of the transwell for 1 hr at 37° C. HNSCC tumor cells in Matrigel containing rhReg3A or PBS were then placed in the upper compartment at 37° C. After 22 hr, cells on the top were wiped off with cotton swabs. Migrated or invaded cells on the lower surface were stained with 0.05% crystal violet (Fisher Scientific) in 20% methanol, rinsed with PBS, and photographed by bright field microscopy. ImageJ (National Institute of Health, MD) was used to quantify the number of cells that had migrated or invaded across the filters. Results are presented as fold change of the number of migrated/invaded cells to the untreated controls. Experiments were performed in triplicate, and statistical significance assessed using Student's t-test.

Gelatin zymography. Conditioned media from control or Reg3A treated cells were mixed with sample buffer (0.03% bromophenol blue, 0.4 mol/l Tris-HCL pH 7.4, 20% glycerol, 5% SDS) and separated on 10% Zymogram Precast Gels (Bio-Rad). After electrophoresis, gels were washed for 1 hr in renaturation buffer (Bio Rad) at room temp for 30 min with gentle shaking. Gels were then incubated at 37° C. with developing buffer to determine enzyme activity, stained with Coomassie Blue (Thermo) and destained with methanol: acetic acid (50:10). Activities of pro-MMP2 and MMP-2 were quantified at 66 and 62 kDA, respectively. Activity of MMP-9 represented a lucid band at 92 kDa.

Quantitative RT-PCR (qRT-PCR) analysis. PDA tumors and normal pancreata were homogenized in 600m1 Buffer RLT Plus from the Qiagen AllPrep RNA/DNA extraction kit, and centrifuged at 18,000g for 3 minutes before total RNA was extracted, following the manufacturer's instructions. RNA concentrations and purity were assessed using the GE NanoVue spectrophotometer. cDNA was synthesized using the Maxima First Strand cDNA synthesis kit for RT-qPCR (Thermo Fisher), using the manufacturer's suggested protocol. Reg3A and Reg3G mRNA expression was quantified using the Taqman Fast Advanced Master Mix (Thermo Fisher) and predesigned Taqman probes. cDNA was diluted tenfold and 5 μl was added to a total reaction volume of 20 μl before performing qRT-PCR according to manufacturer recommended cycling conditions on a BioRad CFX-96 Touch qPCR machine. Gene expression levels were normalized against 18srRNA levels. Taqman assay numbers: Reg3A-Hs00170171_m1, Reg3G-Hs01595406_g1, 18S rRNA-Hs99999901_s1. For HGF mRNA quantitation, cells were treated with Reg3A for 24 h and next quenched with 1 ml Trizol reagent (Thermo Fisher) before RNA extraction according to manufacturer's instructions. cDNA was prepared from total RNA as above, and HGF was quantitated as part of the Cell Motility Prime PCR plate from Bio-Rad using SsoAdvanced (Bio-Rad) according to manufacturer's instructions.

Cell viability assay. Cell lines were plated at 2×10⁴ cells per well in triplicates in 96-well plates containing 10% FBS overnight, then treated with the respective agents for 48 hr in 2.5% FBS media. Cell viability was measured using CytoTox-Glo Cytotoxicity Assay (Promega, WI), according to the manufacturer's protocol. Luminescence counts were read in a Synergy HTX Multi-Mode Reader (BioTek) with the recommended settings.

Human serum Reg3A and sEcad measurements. Serum samples were obtained from patients with histologically confirmed HNSCC from whom pretreatment serum samples were collected prior to surgery or the administration of chemotherapy. The analysis group consisted of 27 males (range 47-89 years) and 33 females (range 44-95). Control sera were obtained from 27 healthy probands. Commercial ELISA kits for secreted Reg3A (R&D Systems, MN) and sEcad (Takara, Shuzo, Japan), together with the magnetic Luminex assay (R&D Systems) for which Reg3A was one of the analyte-specific antibodies, were used to quantify concentrations in control and HNSCC patient serum, per the manufacturer's instruction. Data were plotted as mean+SD from triplicate experiments.

Ex-vivo nerve explants and 3D cultures. Following CO₂ inhalation, rats were euthanized by thoracotomy. Bilateral sciatic nerves were resected using micro-surgical instruments immediately after sacrifice, and transferred to a 6 cm dish containing DMEM/F12 media. Nerves were chopped into small pieces and placed in matrigel, in the presence or absence of rhReg3A for 1 h at 37° C. For co-culture experiments, patient-derived HNSCC cancer-associated fibroblasts (CAFs) were seeded in the lower chamber of the wells at 37° C. After 1 h, HNSCC cells were added to the top wells and maintained for 4-8 days. Media were replaced with fresh media containing Reg3A or PBS every 48-72 hr. Cells were then fixed in 10% formalin, paraffin-embedded, cut into 5 μM sections, and stained with Hematoxylin & Eosin (H&E).

Human tissue samples. This study was approved by the University of Kentucky Institutional Review Board (IRB). All patients provided written informed consent for review of patient medical records, that included, but was not limited to detail regarding past medical histories, medications, as well as demographic, pathologic, and treatment information.

Tissue digestion for CAF isolation. Tumors were harvested, minced into fine pieces (approximately 1 mm³) using scissors and razor blades, and digested in collagenase in complete media at 37° C. Samples were washed with PBS, and resuspended in DMEM/F12 supplemented with 10% FBS, 5 μg/mL insulin, 5 ng/mL FGF, 1 μg/ml hydrocortisone NEAA (100×), L-glutamine (7.5 mM), ascorbic acid (50 m/ml) and penicillin-streptomycin-amphotericin B. All studies were done on cells maintained in culture for less than ten passages.

Statistical Analysis. Statistical significance between groups was determined using Student's t-test. P values<0.05 were considered to indicate statistical significance. All statistical analyses were performed using Prism 7 for Mac OS X (GraphPad Software, Inc.).

### Example 1—Targeting Reg3A/Reg Family Members and Related Methods for Cancer Therapy

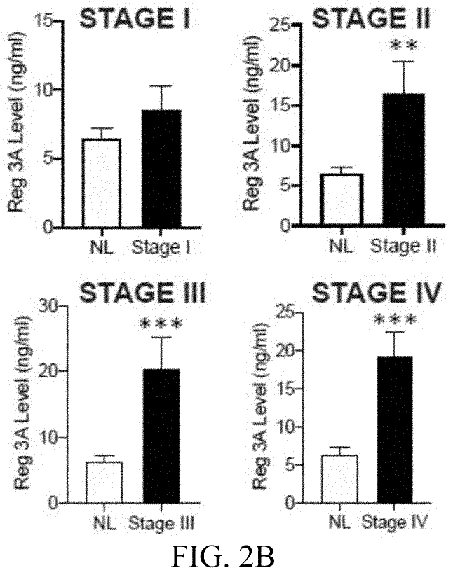

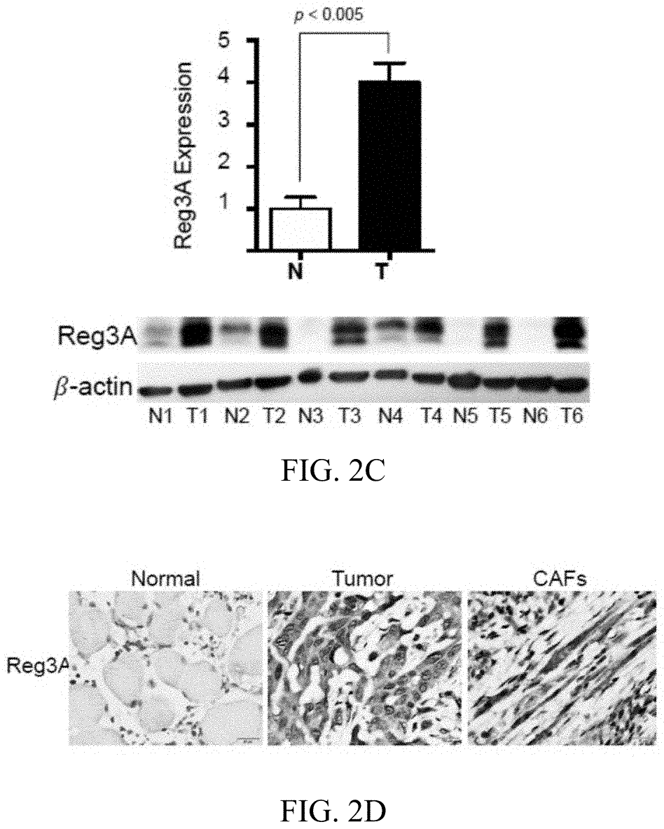

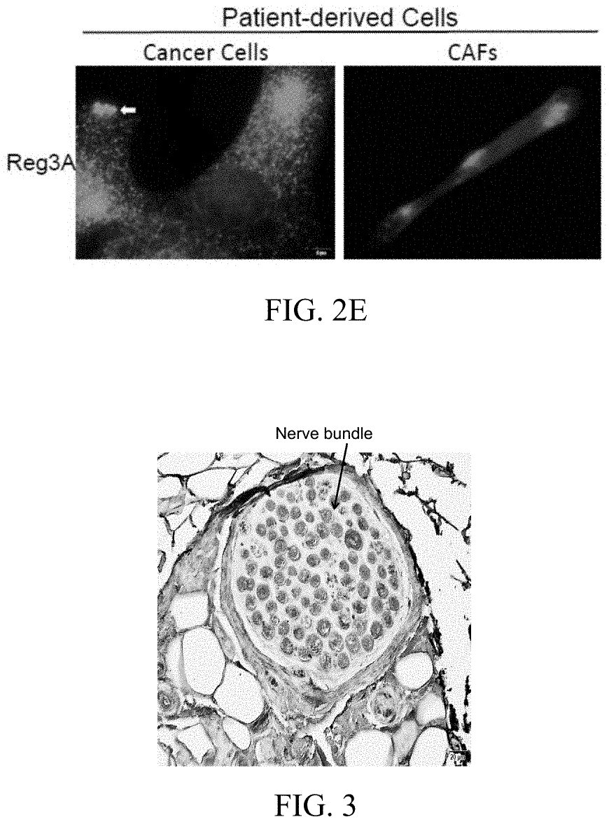

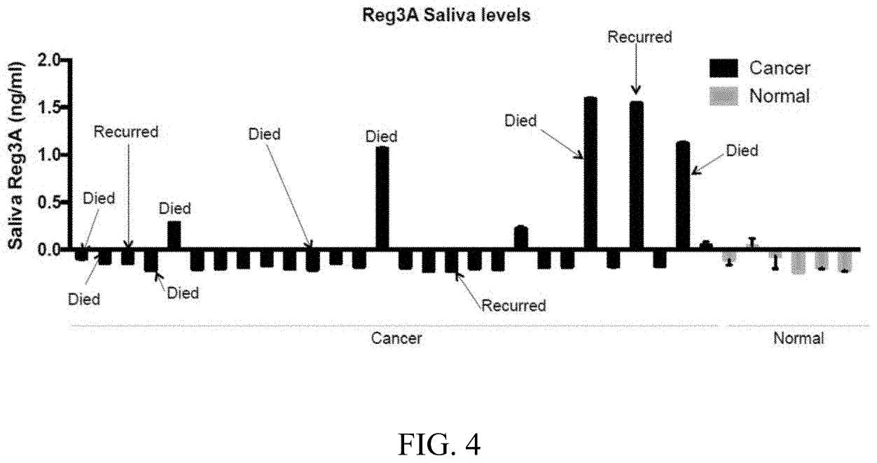

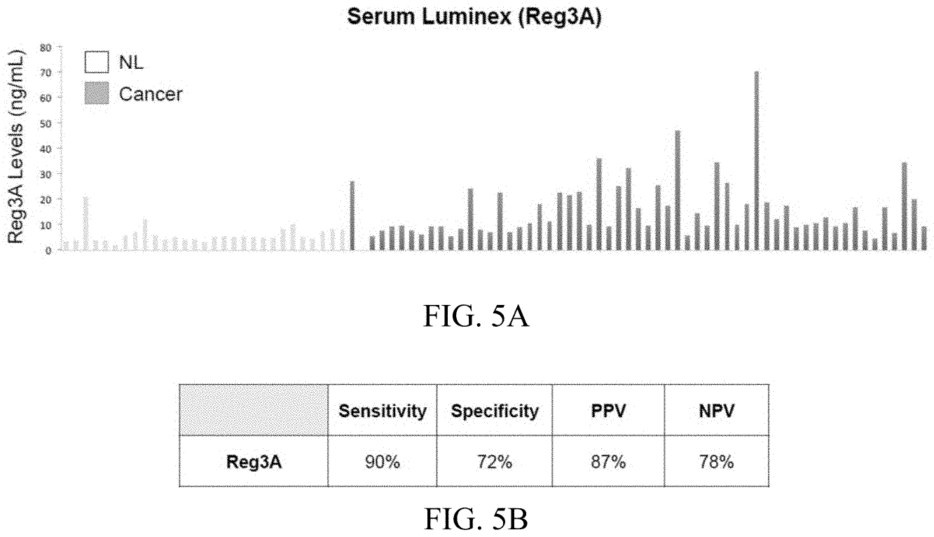

1001211 The following example is based on work with Reg3A and, more specifically, the compositions and methods in the following example relate, at least in part, to a novel finding demonstrating that Reg3A is significantly elevated in HNSCC (oral cancer) patient serum and stage II-IV cancers (Reg3A-specific ELISA), tumors, patient-derived cells (tumors and cancer-associated fibroblasts) (FIGS. 2A-2E) and is expressed in tumor tissue nerve bundles (FIG. 3). This increase in serum Reg3A levels was further validated using the Luminex xMAP multi-analyte profiling technology (FIG. 5). Of note, the findings further suggest that salivary Reg3A levels can predict more aggressive cancers, as they were elevated in 50% and 33.33% of patients that either died or recurred, respectively (FIG. 4). Of note, one patient who died from a post-operative heart attack was excluded. One of the four patients that had a swelling in the neck (recurrence), yet died from cardiac and pulmonary complications, was included.

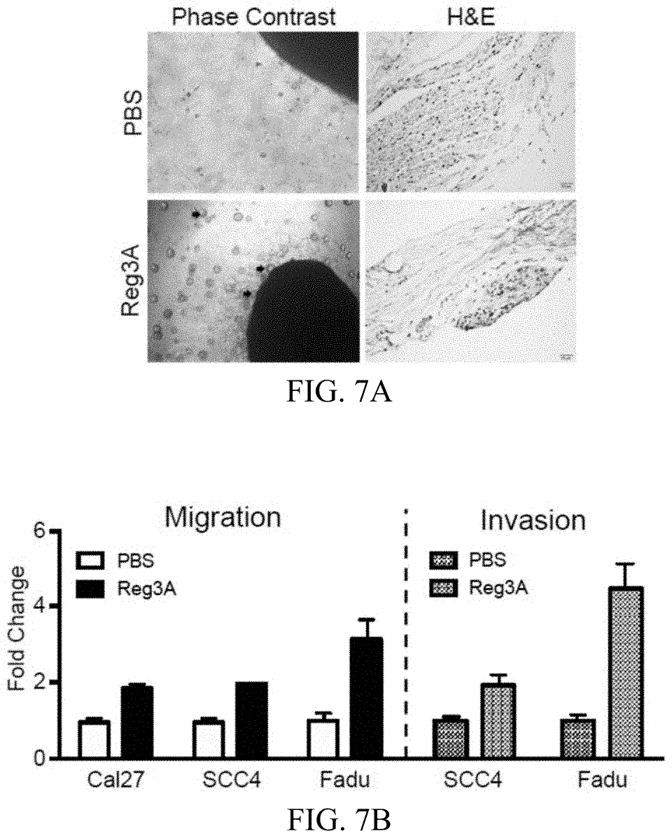

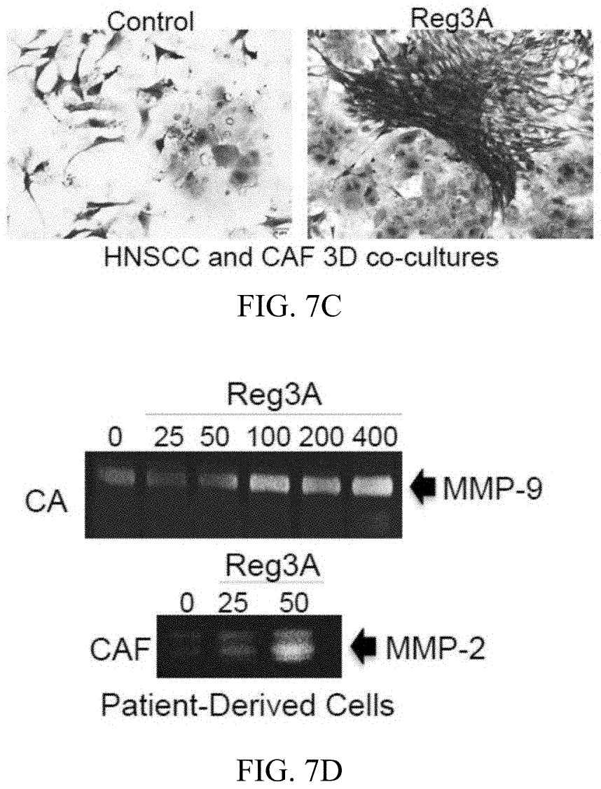

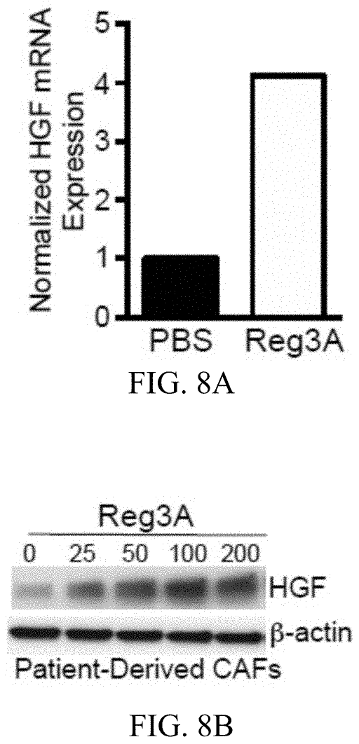

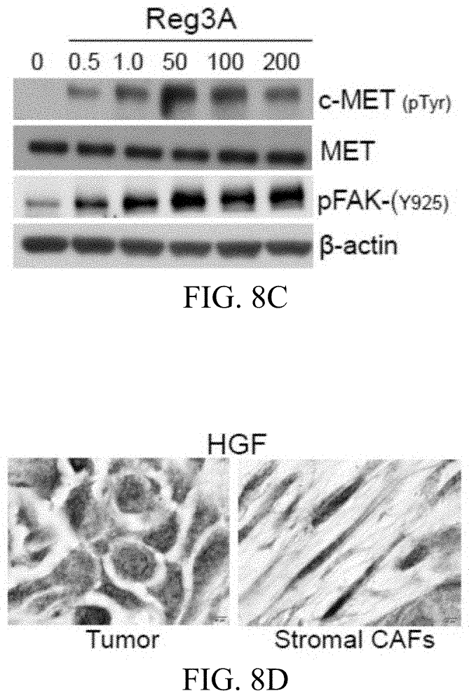

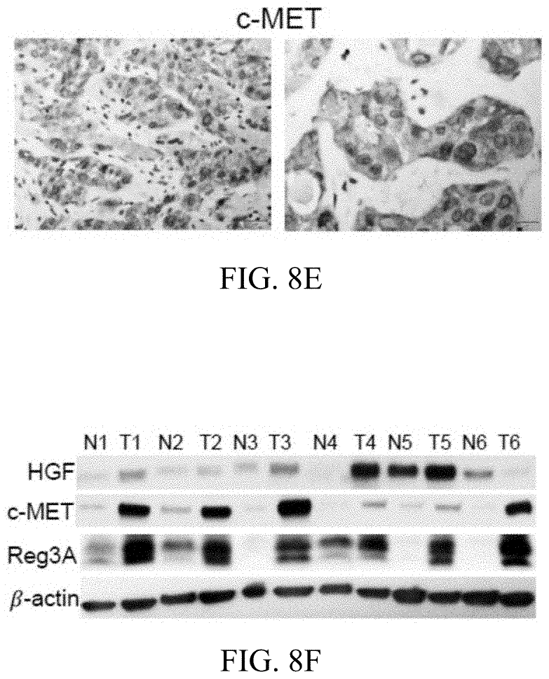

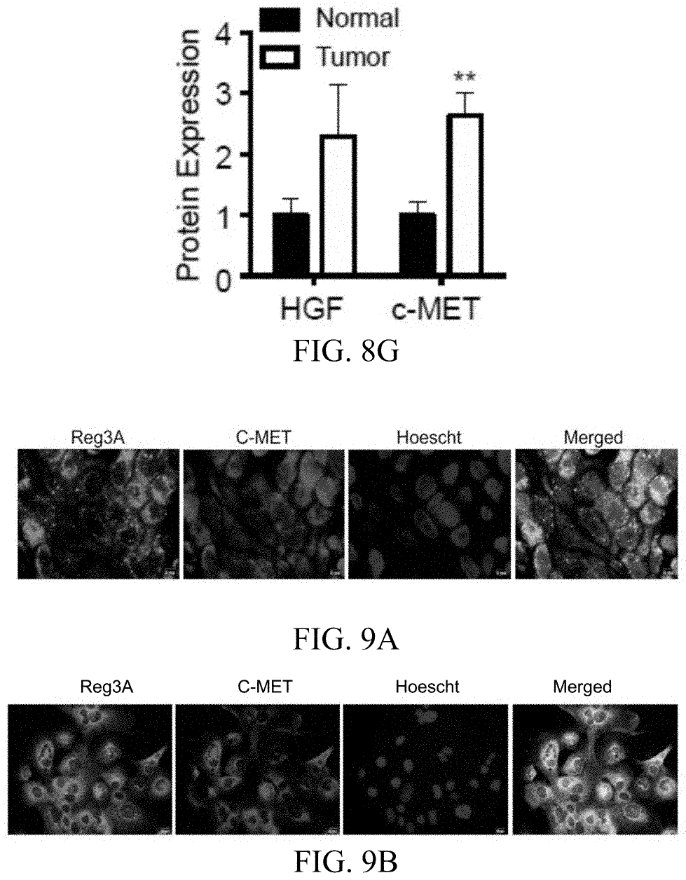

Moreover, the data shows that Reg3A enhances HNSCC perineural invasion (PNI) in rat sciatic nerve explants ex vivo and migration and invasion in multiple HNSCC cell lines in monolayer cultures in vitro (FIGS. 7A-7B). Intriguingly, Reg3A-stimulated patient-derived CAFs encircle and physically contact nests of malignant tumor cells in 3-dimensional (3D) tumor/CAF co-culture systems—mimicking tumor cell-CAF interactions in human tumors (FIG. 7C). In line with these findings, both cell types increased matrix metalloproteinase (MMP2 and MMP-9) activity in response to Reg3A, a step in extracellular matrix breakdown and invasion (FIG. 7D). This was significant, as functional changes in stromal cells and MMP release have been linked to the metastatic potential and chemo-resistance in many solid tumors, including HNSCCs. Mechanistically, the data demonstrate that Reg3A enhances HGF mRNA and protein expression and activates the c-MET-FAK signaling axis (FIGS. 8A-8E). Intriguingly, enhanced c-MET and to a lesser extent HGF expression in the HNSCC tumors versus distal normal specimens (FIGS. 8F-8G) correlated with enhanced Reg3A expression levels (five of the six tumors) and immunoflourescent (IF) staining of Reg3A and c-MET shows some degree of overlap between these proteins in the HNSCC tumors and patient-derived cells (FIGS. 9A-9B), suggesting a possible PPI. Taken together, these findings indicated that Reg3A was a key oncogenic driver and novel target for therapeutic intervention.

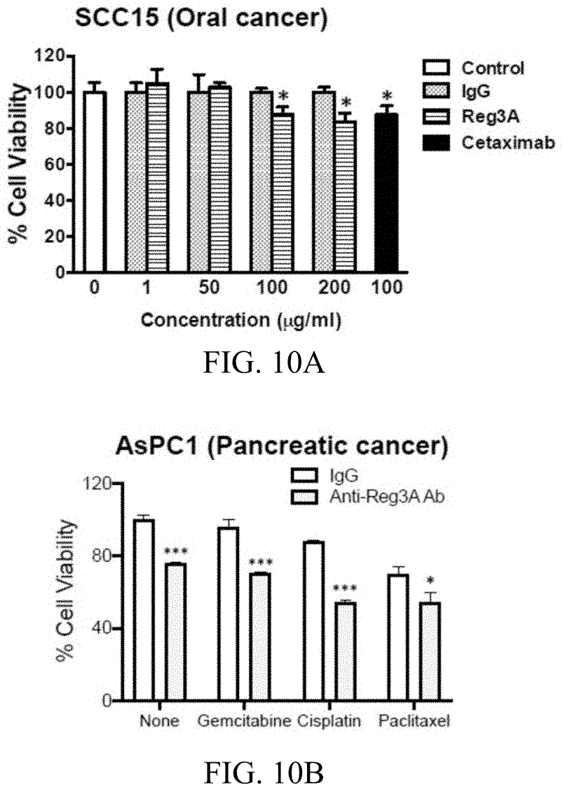

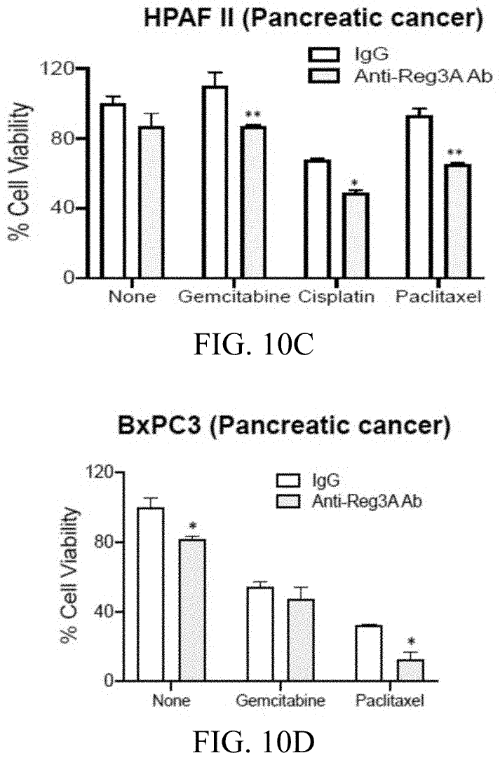

Along these lines, the data further demonstrate that targeting Reg3A with an antibody (Human Reg3A monoclonal antibody (R&D Systems Cat # MAB5965) E. coli-derived recombinant human Reg3A; Glu27-Asp175; Accession # Q06141) resulted in decreased viability of SCC15 HNSCC cancer cells in monolayer cultures [Cell titer Glo; 100% viability for IgG controls (100 and 200 ug/ml) vs. 87.68% and 83.6% (100 and 200 ug/mL respectively)] (FIG. 10A). Notably, this effect on viability was comparable to equal doses of the FDA-approved biologic Cetuximab (viability 87.6% at 100 ug/ml). Of note, these studies were performed at one time point and with single versus repetitive dosing.

### Example 2—Reg3A And Family Members As Biomarkers For Cancer

As shown in FIGS. 5A-5B, it was further determined that enhanced Reg3A levels were present in the serum of HNSCC (Oral Cancer) patients compared to normal subjects. In particular, it was observed that in analyzing Reg3A levels in the serum of 15 normal healthy controls and 23 oral cancer patients based on the Luminex xMAP multi-analyte profiling technology, it was possible to predict with a sensitivity of 91.3% and a specificity of 87.5% that if a patient had a specific cut-off level of Reg3A (>5.5 ng/mL) in their blood (serum), that patient was 73 times (7300 percent) more likely to have cancer. Moreover, it was found that Reg3A levels were significantly increased compared to controls, and that the odds ratio indicated that an individual was 73 times more likely to have oral cancer if they have a level of Reg3A in their blood that is higher than an MFI reading of 1910 (5.5 ng/mL). Additionally, with outcomes according to cut off of a 1910 MFI (5.5 ng/mL) score, the sensitivity of this test was 91.3% with 21 cancer diagnoses out of 23 that would pass the cutoff. The specificity of this test was 87.5% with 14 non-cancer diagnoses out of 16 that are under the cutoff. The false positive rate was 12.5% since there were 2 patients whose MFI score suggests they would have cancer, but did not. The false negative rate was 8.7% since there were 2 patients whose MFI score suggests they would not have cancer, but did. Of those individuals that tested positive, 45% were diagnosed in early stages of cancer and 55% in late stages of disease

### Example 3—Reg3A, Reg Related Family Members And Soluble E-Cadherin (Secad) As Biomarkers For Cancer

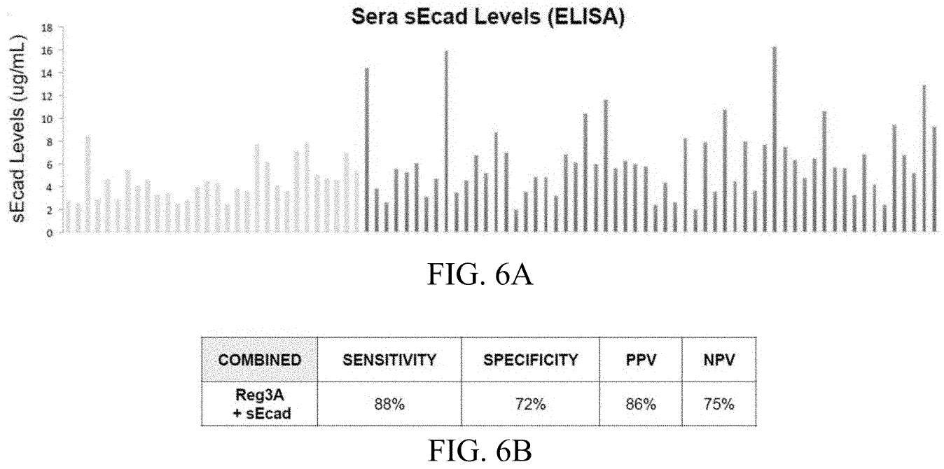

As shown in FIGS. 6A-6B, it was further determined that increased Reg3A together with sEcad represents a biomarker signature panel for HNSCCs (Oral Cancer). In particular, sEcad serum levels in 31 HNSCC cancer patients and 14 healthy controls was measured by ELISA, and the sEcad ELISA was combined with the Reg3A Luminex data, it was possible to predict with a sensitivity of 92% and a specificity of 92.86% that if a patient had a specific cut-off level of Reg3A (5,466 pg/mL) and sEcad (1.99 μg/mL) in their blood (serum), that patient was 12.9 times (1,288 percent) more likely to have cancer. Moreover, as shown in FIG. 10B, the odds ratio indicated that an individual is 12.9 times more likely to have HNSCC (oral cancer) if they have a level of Reg3A and sEcad in their blood that is higher than those values. The sensitivity of this test was 92% with 23 cancer diagnoses out of 25 that would pass the cutoff. The specificity of this test was 92.86% with 13 non-cancer diagnoses out of 14 that are under the cutoff. The false positive rate was 15.38% since there were 2 patients whose values suggests they would have cancer, but did not. The false negative rate was 4.17% since there was 1 patient whose value suggests they would not have cancer, but did.

### Example 4—Reg3A Antibodies for Pancreatic Cancer Therapy

As shown in FIGS. 10B-10D, it was also determined that targeting Reg3A with an anti-Reg3A antibody resulted in decreased cancer cell viability in a number of pancreatic cell lines and that the cancer cell viability was further decreased by the addition of low doses of standard chemotherapy agents. These pancreatic cell lines included AsPC1(P18), HPAF II (P25) and BxPC3 (P10) cells, where the cells were treated with 200 μg/ml of mouse IgG or anti-human Reg3A antibody (R&D, cat # MAB5965), either alone or combined with gemcitabine (0.2 cisplatin (5 μg/ml), or paclitaxel (0.5 μM) for 48 hours. Cell viability was then measured by CytoTox-Glo Cytotoxicity Assay (Promega,cat # G9292).

All publications, patents, and patent applications mentioned in this specification are herein incorporated by reference, including the references set forth in the following list:

### REFERENCES

1. American Cancer Society. (2018). Cancer Facts & Figures.
2. Berx, G., Van Roy, F. (2001). The E-cadherin/catenin complex: an important gatekeeper in breast cancer tumorigenesis and malignant progression. Breast Cancer Res. 3, 289-293.
3. Birchmeier, C.; Birchmeier, W.; Gherardi, E.; Vande Woude, G. F. (2003). Met, metastasis, motility and more. Nat. Rev. Mol. Cell Biol. 4, 915-25.
4. Brouxhon, S., Kyrkanides, S., O'Banion, M. K., Johnson, R., Pearce, D. A., Centola, G. M., Miller, J. N., McGrath, K. H., Erdle, B., Scott, G., et al. (2007). Sequential down-regulation of E-cadherin with squamous cell carcinoma progression: loss of E-cadherin via a prostaglandin E2-EP2 dependent posttranslational mechanism. Cancer Res. 67, 7654-7664.
5. Brouxhon, S. M., Kyrkanides, S., Teng, X., O'Banion, M. K., Clarke, R., Byers, S., Ma, L. (2014a). Soluble-E-cadherin activates HER and IAP family members in HER2+ and TNBC human breast cancers. Mol Carcinog. 53, 893-906.
6. Brouxhon, S. M., Kyrkanides, S., Teng, X., Athar, M., Ghazizadeh, S., Simon, M., O'Banion, M. K., Ma, L. (2014b) Soluble E-cadherin: a critical oncogene modulating receptor tyrosine kinases, MAPK and PI3K/Akt/mTOR signaling. Oncogene. 33, 225-35.
7. Capello, M., Bantis, L. E., Scelo, G., Zhao, Y., Li, P., Dhillon, D. S., Patel, N. J., Kundnani, D. L., Wang, H., Abbruzzese, J. L., et al (2007). Sequential Validation of Blood Based Protein Biomarker Candidates for Early-Stage Pancreatic Cancer. J. Natl. Cancer Inst. 109, djw266.
8. Cavallaro, U. Christofori, G. (2004). Cell adhesion and signalling by cadherins and Ig-CAMs in cancer. Nat. Rev. Cancer. 4,118-32.
9. Chappuis-Flament, S., Wong, E., Hicks, L. D., Kay, C. M., Gumbiner, B. M. (2001) Multiple cadherin extracellular repeats mediate homophilic binding and adhesion. J. Cell Biol. 54, 231-243.
10. Chen, R., Pan, S., Yi, E. C., Donohoe, S., Bronner, M. P., Potter, J. D., Goodlett, D. R., Aebersold, R., Brentnall, T. A. (2006) Quantitative proteomic profiling of pancreatic cancer juice. Proteomics. 6, 3871-3879.
11. Chen, Z. F., Huang, Z. M., Xue, H. B., Lin, XQ., Chen, R. P., Chen, M. J., Jin, R. F. (2017). REG3A promotes the proliferation, migration, and invasion of gastric cancer cells. Onco. Targets Ther. 10, 2017-2023.
12. Cerwenka, H. Aigner, R. Bacher, H. Werkgartner, G. El-Shabrawi, A. Quehenberger, F. Mischinger, H. J. (2001). Pancreatitis-associated protein (PAP) in patients with pancreatic cancer. Anticancer Res. 21, 1471-1474.
13. Choi, B., Suh, Y., Kim, W. H., Christa, L., Park, J., Bae, C. D. (2007) Downregulation of regenerating islet-derived 3 alpha (REG3A) in primary human gastric adenocarcinomas. Exp. Mol. Med. 39, 796-804.
14. Collins, D. C.; Sundar, R.; Lim, J. S. J.; Yap, T. A. (2017). Towards Precision Medicine in the Clinic: From Biomarker Discovery to Novel Therapeutics. Trends Pharmacol. Sci. 38, 25-40.
15. Cortesina, G.; Martone, T.; Galeazzi, E.; Olivero, M.; De Stefani, A.; Bussi, M.; Valente, G.; Comoglio, P. M.; Di Renzo, M. F. (2000). Staging of head and neck squamous cell carcinoma using the MET oncogene product as marker of tumor cells in lymph node metastases. Int. J. Cancer. 89, 286-292.
16. De Wever, O., Derycke, L., Hendrix, A., De Meerleer, G., Godeau, F., Depypere, H., Bracke, M. (2007) Soluble cadherins as cancer biomarkers. Clin. Exp. Metastasis 24, 685-697.
17. Faca, V. M., Song, K. S., Wang, H., Zhang, Q., Krasnoselsky, A. L., Newcomb, L. F. et al. (2008). A mouse to human search for plasma proteome changes associated with pancreatic tumor development. PLoS. Med. 5, e123.
18. Galeazzi, E.; Olivero, M.; Gervasio, F. C.; De Stefani, A.; Valente, G.; Comoglio, P. M.; Di Renzo, M. F.; Cortesina, G. (1997). Detection of MET oncogene/hepatocyte growth factor receptor in lymph node metastases from head and neck squamous cell carcinomas. Eur. Arch. Otorhinolaryngol. 254, S138-S143.
19. Gherardi, E.; Birchmeier, W.; Birchmeier, C.; Vande Woude, G. (2012). Targeting MET in cancer: rationale and progress. Nat. Rev. Cancer. 12, 89-103.
20. Loncle, C., Bonjoch, L., Folch-Puy, E., Lopez-Millan, M. B., Lac, S., Molejon, M. I., Chuluyan, E., Cordelier, P., Dubus, P., Lomberk, G., Urrutia, R., Closa, D., Iovanna, J. L. (2015). IL17 Functions through the Novel REG3β-JAK2-STAT3 Inflammatory Pathway to Promote the Transition from Chronic Pancreatitis to Pancreatic Cancer. Cancer Res. 75, 4852-4862.
21. López-Verdin, S., Soto-Avila, J. J., Zamora-Perez, A. L., Lazalde-Ramos, B. P., Martinez-Fierro, M. L., Gonzalez-Gonzalez, R., Molina-Frechero, N., Isiordia-Espinoza, M. A., Bologna-Molina, R. Patients with advanced oral squamous cell carcinoma have high levels of soluble E-cadherin in the saliva. Med. Oral Patol. Oral Cir. Bucal 22, e694-e701.
22. Ma, P. C., Maulik, G., Christensen, J., Salgia, R. (2003). c-Met: structure, functions and potential for therapeutic inhibition. Cancer Metastasis Rev. 22, 309-25.
23. Masui, T., Ota, I., Itaya-Hironaka, A., Takeda, M., Kasai, T., Yamauchi, A., Sakuramoto-Tsuchida, S., Mikami, S., Yane, K., Takasawa, S., Hosoi, H. (2013). Expression of REG III and prognosis in head and neck cancer. Oncol. Rep. 30, 573-578.
24. Matsumoto, K.; Matsumoto, K.; Nakamura, T.; Kramer, R. H. (1994). Hepatocyte growth factor/scatter factor induces tyrosine phosphorylation of focal adhesion kinase (p125FAK) and promotes migration and invasion by oral squamous cell carcinoma cells. J. Biol. Chem. 269, 31807-31813.
25. Matsumura, N., Zembutsu, H., Yamaguchi, K. Sasaki, K. Tsuruma, T. Nishidate, T., Denno, R., Hirata, K. (2011). Identification of novel molecular markers for detection of gastric cancer cells in the peripheral blood circulation using genome-wide microarray analysis. Exp. Ther. Med. 2, 705-713.
26. Mendonsa, A. M., Na, T. Y., Gumbiner, B. M., (2018). E-cadherin in contact inhibition and cancer. Oncogene. 37, 4769-4780.
27. Miyashita, H., Nakagawara, K., Mori, M., Narushima, Y., Noguchi, N., Moriizumi, S., Takasawa, S., Yonekura, H., Takeuchi, T., Okamoto, H. (1995). Human REG family genes are tandemly ordered in a 95-kilobase region of chromosome 2p12. FEBS Lett. 377, 429-33.
28. Morello, S.; Olivero, M.; Aimetti, M.; Bernardi, M.; Berrone, S.; Di Renzo, M. F.; Giordano, S. (2001). MET receptor is overexpressed but not mutated in oral squamous cell carcinomas. J. Cell Physiol. 189, 285-90.
29. Motoo, Y., Watanabe, H., Yamaguchi, Y., Xie, M. J., Mouri, H., Ohtsubo, K., Okai, T., Wakabayashi, T., Sawabu, N. (2001). Pancreatitis-associated protein levels in pancreatic juice from patients with pancreatic diseases. Pancreatology 1, 43-47.
30. Nass, S. J., Rothenberg, M. L., Pentz, R., Hricak, H., Abernethy, A., Anderson, K., Gee, A. W., Harvey, R. D., Piantadosi, S., Bertagnolli, M. M., Schrag, D., Schilsky, R. L. (2018). Accelerating anticancer drug development—opportunities and trade-offs. Nat. Rev. Clin. Oncol. 15, 777-786.
31. Nata, K., Liu, Y., Xu, L., Ikeda, T., Akiyama, T., Noguchi, N., Kawaguchi, S., Yamauchi, A., Takahashi, I., Shervani, N. J., Onogawa, T., Takasawa, S., Okamoto, H. (2004). Molecular cloning, expression and chromosomal localization of a novel human REG family gene, REG III. Gene. 340, 161-170.
32. Nigri, J., Gironella, M., Bressy, C., Vila-Navarro, E., Rogues, J., Lac, S., Bontemps, C., Kozaczyk, C., Cros, J., Pietrasz, D., Marechal, R., et al. (2017). PAP/REG3A favors perineural invasion in pancreatic adenocarcinoma and serves as a prognostic marker. Cell. Mol. Life Sci. 74, 4231-4243.
33. Papaleo, E., Gromova, I., Gromov, P. (2017). Gaining insights into cancer biology through exploration of the cancer secretome using proteomic and bioinformatic tools. Expert Rev. Proteomics. 14, 1021-1035.
34. Qiu, Y. S., Liao, G. J., Jiang, N. N. (2018). REG3A overexpression suppresses gastric cancer cell invasion, proliferation and promotes apoptosis through PI3K/Akt signaling pathway. Int. J. Mol. Med. 41, 3167-3174.
35. Repetto, O., De Paoli, P., De Re, V., Canzonieri, V., Cannizzaro, R. (2014). Levels of soluble E-cadherin in breast, gastric, and colorectal cancers. Biomed. Res. Int. 2014, 408047.
36. Rosty, C., Christa, L., Kuzdzal, S., Baldwin, W. M., Zahurak, M. L., Carnot, F., Chan, D. W., Canto, M., Lillemoe, K. D., Cameron, J. L., Yeo, C. J., Hruban, R. H., Goggins, M. Identification of hepatocarcinoma-intestine-pancreas/pancreatitis-associated protein I as a biomarker for pancreatic ductal adenocarcinoma by protein biochip technology. Cancer Res. 62, 1868-1875.
37. Rothenberger, N. J.; Stabile, L. P. (2017). Hepatocyte Growth Factor/c-Met Signaling in Head and Neck Cancer and Implications for Treatment. Cancers (Basel). 9, E39.
38. Sulzmaier, F. J.; Jean, C.; Schlaepfer, D. D. (2014). FAK in cancer: mechanistic findings and clinical applications. Nat. Rev. Cancer. 14, 598-610.
39. Teng, X., Ma, L., Kyrkanides, S., Raja, V., Trochesset, D., Brouxhon, S. (2015). Modulation of RTK by sEcad: a putative mechanism for oncogenicity in oropharyngeal SCCs. Oral Dis. 21, 185-94.
40. The UniProt Consortium. (2017). UniProt: the universal protein knowledgebase. Nucleic Acids Res. 45, D158-D169.
41. Wang, G. Zhao, X. Li, D. C. (2015). Expression of HIP/PAP in hepatocellular carcinoma and effect of siRNA on migration and invasion in HCC cells. Asian Pac. J. Trop. Med. 8, 848-54.
42. Wedge, D. C.; Gundem, G.; Mitchell, T.; Woodcock, D. J.; Martincorena, I.; Ghori, M.; Zamora, J.; Butler, A.; Whitaker, H.; Kote-Jarai, Z.; et al. (2018). Sequencing of prostate cancers identifies new cancer genes, routes of progression and drug targets. Nat. Genet. 50, 682-692.
43. Yazdani, J., Ghavimi, M. A., Jabbari Hagh, E., Ahmadpour F. (2018). The Role of E-cadherin as a Prognostic Biomarker in Head and Neck Squamous Carcinoma: A Systematic Review and Meta-Analysis. Mol. Diagn. Ther. [Epub ahead of print].

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for diagnosing a cancer in a subject, comprising:
(a) providing a biological sample from the subject;
(b) determining an amount of Reg3A in the sample; and
(c) diagnosing the subject as having cancer or a risk thereof if there is a measurable difference in the amount of the Reg3A in the biological sample as compared to a control level of the Reg3A.

2. The method of claim 1, further comprising administering to the diagnosed subject an effective amount of an agent capable of affecting an expression level or activity of Reg3A.

3. The method of claim 2, wherein the agent is an anti-Reg3A antibody.

4. The method of claim 2, further comprising administering an effective amount of a chemotherapeutic agent to the subject.

5. The method of claim 1, wherein the cancer is a head and neck cancer.

6. The method of claim 1, wherein the head and neck cancer is an oral cancer.

7. The method of claim 1, wherein the biological sample comprises blood, plasma, serum, saliva, exosomes, urine, cerebrospinal fluid, ascites fluid, a tissue sample, a tumor sample, or combinations thereof.

8. The method of claim 1, wherein the subject is human.

9. The method of claim 1, wherein the subject has cancer.

10. The method of claim 1, wherein the cancer is selected from the group consisting of head and neck cancer, colorectal cancer, pancreatic cancer, ovarian cancer, cervical cancer, breast cancer, renal cell cancer, non-small cell lung cancer, small cell lung cancer, mesothelioma, gastric cancer, esophageal cancer, bladder cancer, melanoma, thyroid cancer, prostate cancer, leukemia, lymphoma, myelomas, mycoses fungoids, and merkel cell cancer.

11. The method of claim 10, wherein the cancer is pancreatic cancer.

12. The method of claim 1, wherein determining the amount in the sample of the Reg3A comprises determining the amount in the sample of the Reg3A using mass spectrometry (MS) analysis, immunoassay analysis, or both.

13. The method of claim 1, wherein determining the amount in the sample of the Reg3A comprises determining the amount of Reg3A protein in the sample.

14. The method of claim 1, wherein determining the amount in the sample of the Reg3A comprises determining the amount of Reg3A nucleic acid expression in the sample.

15. The method of claim 1, wherein the sample is obtained prior to a treatment for the cancer.

16. The method of claim 1, further comprising selecting a treatment or modifying a treatment for the cancer based on the determined amount of the Reg3A.

17. The method of claim 1, further comprising determining an amount in the sample of soluble E-cadherin (sEcad).

18. The method of claim 1, further comprising determining an amount in the sample of one or more additional Reg family members.

19. A method for determining whether to initiate or continue prophylaxis or treatment of a cancer in a subject, comprising:
(a) providing a series of biological samples over a time period from the subject;
(b) analyzing the series of biological samples to determine an amount in each of the biological samples of Reg3A; and
(c) comparing any measurable change in the amounts of the Reg3A in each of the biological samples to thereby determine whether to initiate or continue the prophylaxis or therapy of the cancer.

20. The method of claim 19, further comprising determining an amount in the sample of soluble E-cadherin (sEcad).

21. The method of claim 19, wherein the series of biological samples comprises a first biological sample collected prior to initiation of the prophylaxis or treatment for the cancer and a second biological sample collected after initiation of the prophylaxis or treatment.

22. The method of claim 19, wherein the series of biological samples comprises a first biological sample collected prior to onset of the cancer and a second biological sample collected after the onset of the cancer.

23. A method for diagnosing a cancer in a subject, comprising:
applying an agent capable of affecting detection of an amount of Reg3A to a biological sample obtained from the subject; and
determining the amount of Reg3A in the sample.

24. The method of claim 23, further comprising:
applying an agent capable of affecting detection of an amount of soluble E-cadherin (sEcad) to the biological sample obtained from the subject; and
determining the amount of sEcad in the sample.

25. A method for treating a cancer, comprising administering to a subject in need thereof an effective amount of an agent capable of affecting an expression level or activity of Reg3A.

26. The method of claim 25, wherein the agent is an anti-Reg3A antibody.

27. The method of claim 25, wherein the cancer is selected from the group consisting of head and neck cancer, colorectal cancer, pancreatic cancer, ovarian cancer, cervical cancer, breast cancer, renal cell cancer, non-small cell lung cancer, small cell lung cancer, mesothelioma, gastric cancer, esophageal cancer, bladder cancer, melanoma, thyroid cancer, prostate cancer, leukemia, lymphoma, myelomas, mycoses fungoids, and merkel cell cancer.

28. The method of claim 25, further comprising administering an effective amount of a chemotherapeutic agent to the subject.

29. The method of claim 25, wherein administering the agent comprises oral administration, intravenous administration, nasal or inhalation administration, intramuscular administration, intraperitoneal administration, transmucosal administration, or transdermal administration.

30. The method of claim 25, further comprising the step of providing a biological sample from the subject and determining an amount of Reg3A, a Reg family member, and/or sEcad in the biological sample, and wherein the effective amount administered to the subject is based on the amount of Reg3A, the Reg family member, and/or the sEcad in the biological sample.
